(19) United States
(12) Patent Application Publication
(10) Pub. No.: US 2023/0357163 A1
(43) Pub. Date: Nov. 9, 2023

(54) SOLID STATE FORMS OF GEFAPIXANT AND PROCESS FOR PREPARATION THEREOF

(71) Applicant: TEVA PHARMACEUTICALS INTERNATIONAL GMBH, Jona (CH)

(72) Inventors: Dijana Skalec Samec, Jastrebarsko (HR); Valentina Travancic, Zagreb (HR)

(21) Appl. No.: 18/026,211

(22) PCT Filed: Sep. 16, 2021
(86) PCT No.: PCT/US21/50614

§ 371 Date: Mar. 14, 2023

Related U.S. Application Data
(60) Provisional application No. 63/105,430, filed on Oct. 26, 2020.
(60) Provisional application No. 63/079,524, filed on Sep. 17, 2020.

(51) Int. Cl. C07D 239/48

(52) U.S. Cl. CPC C07D 239/48; C07B 2200/13

(57) ABSTRACT

The present disclosure encompasses solid state forms of Gefapixant, in embodiments crystalline polymorphs or salts or co-crystals of Gefapixant, processes for preparation thereof, and pharmaceutical compositions thereof.

### FIELD OF THE DISCLOSURE

The present disclosure encompasses solid state forms of Gefapixant, in embodiments crystalline polymorphs or salts or co-crystals of Gefapixant, processes for preparation thereof, and pharmaceutical compositions thereof.

### BACKGROUND OF THE DISCLOSURE

Gefapixant, 5-(2,4-diamino-pyrimidin-5-yloxy)-4-isopropyl-2-methoxy-benzenesulfonamide, has the following chemical structure:

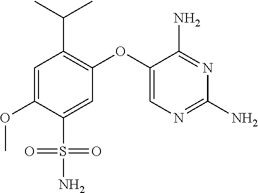 [SMILES: COc1cc(C(C)C)c(Oc2cnc(N)nc2N)cc1S(N)(=O)=O]

Gefapixant is a purinergic P2X3 receptor antagonist, and it is developed for the treatment of chronic cough. Gefapixant is also under clinical investigation as a treatment for asthma, interstitial cystitis, musculoskeletal pain, pelvic pain, and sleep apnea syndrome.

The compound is described in International Publication No. WO 2005/95359. International Publication No. WO 2008/040652 disclosed a sulfonate solvate of Gefapixant. International Publication Nos. WO 2018/118668 and WO 2019/209607 disclose crystalline forms of Gefapixant as well as Gefapixant salts.

Polymorphism, the occurrence of different crystalline forms, is a property of some molecules and molecular complexes. A single molecule may give rise to a variety of polymorphs having distinct crystal structures and physical properties like melting point, thermal behaviors (e.g., measured by thermogravimetric analysis (“TGA”), or differential scanning calorimetry (“DSC”)), X-ray diffraction (XRD) pattern, infrared absorption fingerprint, and solid state (¹³C) NMR spectrum. One or more of these techniques may be used to distinguish different polymorphic forms of a compound.

Different salts and solid state forms (including solvated forms) of an active pharmaceutical ingredient may possess different properties. Such variations in the properties of different salts and solid state forms and solvates may provide a basis for improving formulation, for example, by facilitating better processing or handling characteristics, changing the dissolution profile in a favorable direction, or improving stability (polymorph as well as chemical stability) and shelf-life. These variations in the properties of different salts and solid state forms may also offer improvements to the final dosage form, for instance, if they serve to improve bioavailability. Different salts and solid state forms and solvates of an active pharmaceutical ingredient may also give rise to a variety of polymorphs or crystalline forms, which may in turn provide additional opportunities to assess variations in the properties and characteristics of a solid active pharmaceutical ingredient.

Discovering new solid state forms and solvates of a pharmaceutical product may yield materials having desirable processing properties, such as ease of handling, ease of processing, storage stability, and ease of purification or as desirable intermediate crystal forms that facilitate conversion to other polymorphic forms. New solid state forms of a pharmaceutically useful compound can also provide an opportunity to improve the performance characteristics of a pharmaceutical product. It enlarges the repertoire of materials that a formulation scientist has available for formulation optimization, for example by providing a product with different properties, including a different crystal habit, higher crystallinity, or polymorphic stability, which may offer better processing or handling characteristics, improved dissolution profile, or improved shelf-life (chemical/physical stability). For at least these reasons, there is a need for additional solid state forms (including solvated forms) of Gefapixant or salts or co-crystals thereof.

### SUMMARY OF THE DISCLOSURE

The present disclosure provides crystalline polymorphs or salts or co-crystals of Gefapixant, processes for preparation thereof, and pharmaceutical compositions thereof. These crystalline polymorphs can be used to prepare other solid state forms of Gefapixant, Gefapixant salts or co-crystals and their solid state forms.

The present disclosure also provides uses of the said solid state forms of Gefapixant or salts or co-crystals thereof in the preparation of other solid state forms of Gefapixant or salts or co-crystals thereof.

The present disclosure provides crystalline polymorphs of Gefapixant or salts or co-crystals thereof for use in medicine, including for the treatment of chronic cough, asthma, interstitial cystitis, musculoskeletal pain, pelvic pain, or sleep apnea syndrome, and particularly for the treatment of chronic cough.

The present disclosure also encompasses the use of crystalline polymorphs of Gefapixant or salts or co-crystals thereof of the present disclosure for the preparation of pharmaceutical compositions and/or formulations.

In another aspect, the present disclosure provides pharmaceutical compositions comprising crystalline polymorphs of Gefapixant or salts or co-crystals thereof according to the present disclosure.

The present disclosure includes processes for preparing the above mentioned pharmaceutical compositions. The processes include combining any one or a combination of the crystalline polymorphs of Gefapixant or salts or co-crystals thereof with at least one pharmaceutically acceptable excipient.

The crystalline polymorph of Gefapixant or salts or co-crystals thereof as defined herein and the pharmaceutical compositions or formulations of the crystalline polymorph of Gefapixant or salts or co-crystals thereof may be used as medicaments, such as for the treatment of chronic cough, asthma, interstitial cystitis, musculoskeletal pain, pelvic pain, or sleep apnea syndrome, and particularly for the treatment of chronic cough.

The present disclosure also provides methods of treating Gefapixant or salts or co-crystals thereof, by administering a therapeutically effective amount of any one or a combination of the crystalline polymorphs of Gefapixant or salts or co-crystals thereof of the present disclosure, or at least one of the above pharmaceutical compositions, to a subject suffering from chronic cough, asthma, interstitial cystitis, musculoskeletal pain, pelvic pain, or sleep apnea syndrome, and particularly to a subject suffering from chronic cough, or otherwise in need of the treatment.

The present disclosure also provides uses of crystalline polymorphs of Gefapixant or salts or co-crystals thereof of the present disclosure, or at least one of the above pharmaceutical compositions, for the manufacture of medicaments for treating e.g. chronic cough, asthma, interstitial cystitis, musculoskeletal pain, pelvic pain, or sleep apnea syndrome, and particularly chronic cough.

### BRIEF DESCRIPTION OF THE DRAWINGS

FIG. 1 shows a characteristic X-ray powder diffraction pattern (XRPD) of Gefapixant Form 1;

FIG. 2 shows a characteristic XRPD of a Gefapixant Form 2;

FIG. 3 shows a characteristic XRPD of a Gefapixant Form 4;

FIG. 4 shows a characteristic XRPD of a Gefapixant citrate Form T1;

FIG. 5 shows a characteristic XRPD of a Gefapixant citrate Form E1;

FIG. 6 shows a characteristic XRPD of a Gefapixant maleate Form M1;

FIG. 7 shows a characteristic XRPD of a Gefapixant citrate amorphous Form;

FIG. 8 shows a characteristic XRPD of Gefapixant camsylate Form Ic;

FIG. 9 shows a characteristic XRPD of Gefapixant tosylate Form It;

FIG. 10 shows a characteristic XRPD of Gefapixant hemiedisylate Form Ie;

FIG. 11 shows a characteristic XRPD of Gefapixant edisylate Form IIe;

FIG. 12 shows a characteristic XRPD of Gefapixant malonate Form Im;

FIG. 13 shows a characteristic XRPD of Gefapixant fumarate Form If;

FIG. 14 shows a characteristic XRPD of Crystalline Form L1 of Gefapixant citrate: L-Lysine complex;

FIG. 15 shows a characteristic XRPD of Gefapixant tosylate Form IIt;

FIG. 16 shows a characteristic XRPD of Gefapixant fumarate Form IIf as obtained by example 17;

FIG. 17 shows a characteristic DSC thermogram of a Gefapixant maleate Form M1;

FIG. 18 shows a characteristic TGA thermogram of a Gefapixant maleate Form M1;

FIG. 19 shows a characteristic DSC thermogram of a Gefapixant camsylate Form Ic;

FIG. 20 shows a characteristic TGA thermogram of a Gefapixant camsylate Form Ic;

FIG. 21 shows a characteristic DSC thermogram of a Gefapixant hemiedisylate Form Ie; and

FIG. 22 shows a characteristic TGA thermogram of a hemiedisylate Form Ie.

### DETAILED DESCRIPTION OF THE DISCLOSURE

The present disclosure encompasses solid state forms of Gefapixant, including crystalline polymorphs of Gefapixant or salts or co-crystals thereof, processes for preparation thereof, and pharmaceutical compositions thereof.

Solid state properties of Gefapixant or salts or co-crystals thereof and crystalline polymorphs thereof can be influenced by controlling the conditions under which Gefapixant or salts or co-crystals thereof and crystalline polymorphs thereof are obtained in solid form.

A solid state form (or polymorph) may be referred to herein as polymorphically pure or as substantially free of any other solid state (or polymorphic) forms. As used herein in this context, the expression “substantially free of any other forms” will be understood to mean that the solid state form contains about 20% (w/w) or less, about 10% (w/w) or less, about 5% (w/w) or less, about 2% (w/w) or less, about 1% (w/w) or less, or about 0% of any other forms of the subject compound as measured, for example, by XRPD. Thus, a crystalline polymorph of Gefapixant or salts or co-crystals thereof described herein as substantially free of any other solid state forms would be understood to contain greater than about 80% (w/w), greater than about 90% (w/w), greater than about 95% (w/w), greater than about 98% (w/w), greater than about 99% (w/w), or about 100% of the subject crystalline polymorph of Gefapixant or salts or co-crystals thereof. In some embodiments of the disclosure, the described crystalline polymorph of Gefapixant or salts or co-crystals thereof may contain from about 1% to about 20% (w/w), from about 5% to about 20% (w/w), or from about 5% to about 10% (w/w) of one or more other crystalline polymorph of the same Gefapixant or salts or co-crystals thereof.

Depending on which other crystalline polymorphs a comparison is made, the crystalline polymorphs of Gefapixant or salts or co-crystals thereof of the present disclosure may have advantageous properties selected from at least one of the following: chemical purity, flowability, solubility, dissolution rate, morphology or crystal habit, stability, such as chemical stability as well as thermal and mechanical stability with respect to polymorphic conversion, stability towards dehydration and/or storage stability, low content of residual solvent, a lower degree of hygroscopicity, flowability, and advantageous processing and handling characteristics such as compressibility and bulk density.

A solid state form, such as a crystal form or an amorphous form, may be referred to herein as being characterized by graphical data “as depicted in” or “as substantially depicted in” a Figure. Such data include, for example, powder X-ray diffractograms and solid state NMR spectra. As is well-known in the art, the graphical data potentially provides additional technical information to further define the respective solid state form (a so-called “fingerprint”) which cannot necessarily be described by reference to numerical values or peak positions alone. In any event, the skilled person will understand that such graphical representations of data may be subject to small variations, e.g., in peak relative intensities and peak positions due to certain factors such as, but not limited to, variations in instrument response and variations in sample concentration and purity, which are well known to the skilled person. Nonetheless, the skilled person would readily be capable of comparing the graphical data in the Figures herein with graphical data generated for an unknown crystal form and confirm whether the two sets of graphical data are characterizing the same crystal form or two different crystal forms. A crystal form of Gefapixant or salts or co-crystals thereof referred to herein as being characterized by graphical data “as depicted in” or “as substantially depicted in” a Figure will thus be understood to include any crystal forms of Gefapixant or salts or co-crystals thereof characterized with the graphical data having such small variations, as are well known to the skilled person, in comparison with the Figure.

As used herein, and unless stated otherwise, the term “anhydrous” in relation to crystalline forms of Gefapixant or salts or co-crystals thereof, relates to a crystalline form of Gefapixant or salts or co-crystals thereof which does not include any crystalline water (or other solvents) in a defined, stoichiometric amount within the crystal. Moreover, an “anhydrous” form would generally not contain more than 1% (w/w), of either water or organic solvents as measured for example by TGA.

The term “solvate,” as used herein and unless indicated otherwise, refers to a crystal form that incorporates a solvent in the crystal structure. When the solvent is water, the solvate is often referred to as a “hydrate.” The solvent in a solvate may be present in either a stoichiometric or in a non-stoichiometric amount.

“Co-Crystal” or “Co-crystal” or “co-crystal” as used herein is defined as a crystalline material including two or more molecules in the same crystalline lattice and associated by non-ionic and non-covalent bonds. In some embodiments, the co-crystal includes two molecules which are in natural state.

“Cocrystal former” or “crystal former” as used herein is defined as a molecule that forms a cocrystal with Gefapixant or salts thereof, for example L-Lysine.

As used herein, crystalline Gefapixant citrate: L-Lysine is a distinct molecular species. Crystalline Gefapixant citrate: L-Lysine may be a co-crystal of Gefapixant citrate and L-Lysine.

As used herein, the term “isolated” in reference to crystalline polymorph of Gefapixant or salts or co-crystals thereof of the present disclosure corresponds to a crystalline polymorph of Gefapixant or salts or co-crystals thereof that is physically separated from the reaction mixture in which it is formed.

As used herein, unless stated otherwise, the XRPD measurements are taken using copper Kα radiation wavelength 1.5418 Å. XRPD peaks reported herein are measured using CuK α radiation, λ=1.5418 Å, typically at a temperature of 25±3° C.

As used herein, unless stated otherwise, ¹³C NMR reported herein are measured at 125 MHZ AT A MAGIC ANGLE SPINNING FREQUENCY ω_{R}/2π=11 KHZ, PREFERABLY AT A TEMPERATURE OF AT 293 K±3° C.

As used herein, unless stated otherwise, DSC measurements are obtained by heating from 25-350° C., at a heating rate of 10° C./min, under nitrogen. Preferably about 1-3 mg of sample is used.

As used herein, unless stated otherwise, TGA measurements are obtained by heating from 25-350° C., at a heating rate of 10° C./min, under nitrogen. Preferably about 5-10 mg of sample is used.

A thing, e.g., a reaction mixture, may be characterized herein as being at, or allowed to come to “room temperature” or “ambient temperature”, often abbreviated as “RT.” This means that the temperature of the thing is close to, or the same as, that of the space, e.g., the room or fume hood, in which the thing is located. Typically, room temperature is from about 20° C. to about 30° C., or about 22° C. to about 27° C., or about 25° C.

The amount of solvent employed in a chemical process, e.g., a reaction or crystallization, may be referred to herein as a number of “volumes” or “vol” or “V.” For example, a material may be referred to as being suspended in 10 volumes (or 10 vol or 10V) of a solvent. In this context, this expression would be understood to mean milliliters of the solvent per gram of the material being suspended, such that suspending a 5 grams of a material in 10 volumes of a solvent means that the solvent is used in an amount of 10 milliliters of the solvent per gram of the material that is being suspended or, in this example, 50 mL of the solvent. In another context, the term “v/v” may be used to indicate the number of volumes of a solvent that are added to a liquid mixture based on the volume of that mixture. For example, adding solvent X (1.5 v/v) to a 100 ml reaction mixture would indicate that 150 mL of solvent X was added.

A process or step may be referred to herein as being carried out “overnight.” This refers to a time interval, e.g., for the process or step, that spans the time during the night, when that process or step may not be actively observed. This time interval is from about 8 to about 20 hours, or about 10-18 hours, in some cases about 16 hours.

As used herein, the term “reduced pressure” refers to a pressure that is less than atmospheric pressure. For example, reduced pressure is about 10 mbar to about 50 mbar.

As used herein and unless indicated otherwise, the term “ambient conditions” refer to atmospheric pressure and a temperature of 22-24° C.

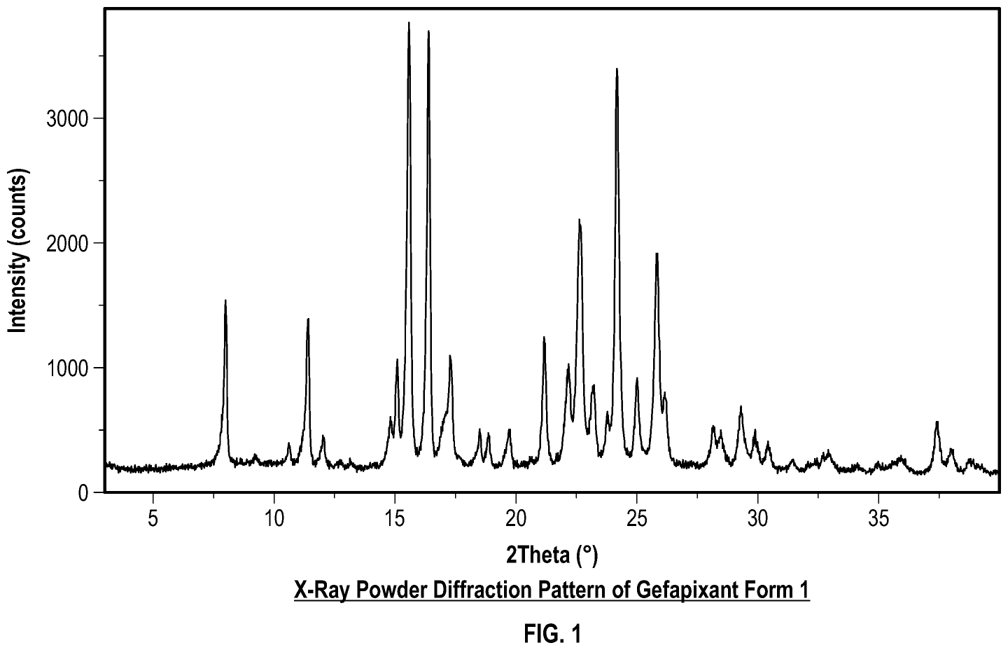

The present disclosure includes a crystalline polymorph of Gefapixant, designated Form 1. The crystalline Form 1 of Gefapixant may be characterized by data selected from one or more of the following: an X-ray powder diffraction pattern substantially as depicted in FIG. 1; an X-ray powder diffraction pattern having peaks at 8.0, 11.4, 15.6, 22.7 and 24.2 degrees 2-theta±0.2 degrees 2-theta; and combinations of these data.

Crystalline Form 1 of Gefapixant may be further characterized by an X-ray powder diffraction pattern having peaks at 8.0, 11.4, 15.6, 22.7 and 24.2 degrees 2-theta±0.2 degrees 2-theta, and also having any one, two, three, four or five additional peaks selected from 15.1, 16.4, 22.2, 25.0 and 25.8 degrees 2-theta±0.2 degrees 2-theta.

Crystalline Form 1 of Gefapixant may be alternatively characterized by an X-ray powder diffraction pattern having peaks at 8.0, 11.4, 15.1, 15.6, 16.4, 22.2, 22.7, 24.2, 25.0, and 25.8 degrees 2-theta±0.2 degrees 2-theta.

In one embodiment of the present disclosure, crystalline Form 1 of Gefapixant is isolated.

Crystalline Form 1 of Gefapixant may be anhydrous form.

Crystalline Form 1 of Gefapixant may be characterized by each of the above characteristics alone or by all possible combinations, e.g., an XRPD pattern having peaks at 8.0, 11.4, 15.6, 22.7 and 24.2 degrees 2-theta±0.2 degrees 2-theta; an XRPD pattern as depicted in FIG. 1, and combinations thereof.

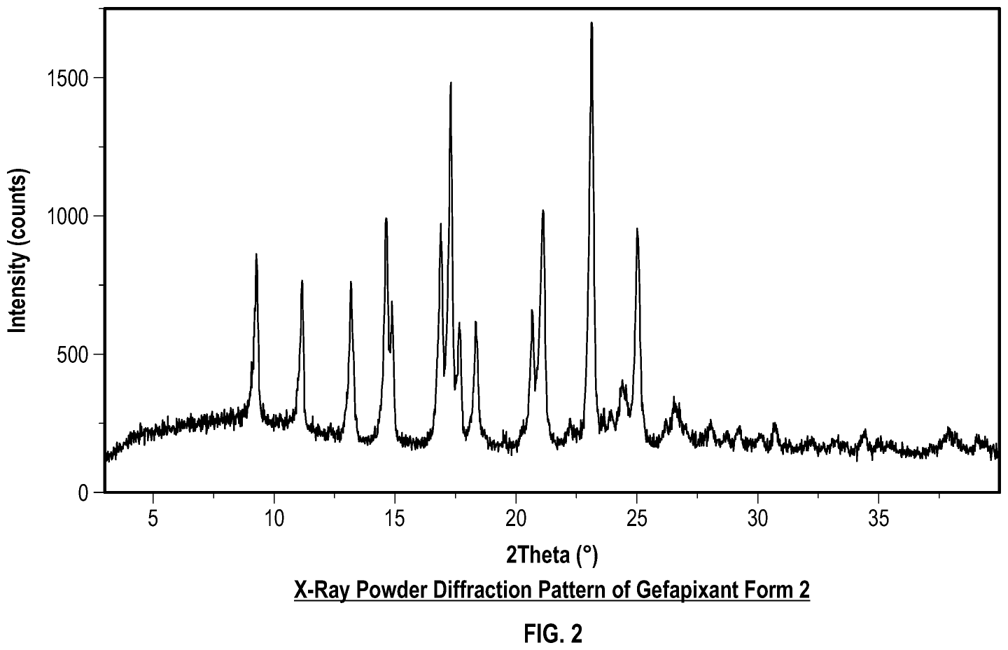

The present disclosure includes a crystalline polymorph of Gefapixant, designated Form 2. The crystalline Form 2 of Gefapixant may be characterized by data selected from one or more of the following: an X-ray powder diffraction pattern substantially as depicted in FIG. 2; an X-ray powder diffraction pattern having peaks at 9.3, 13.2, 14.6, 16.9 and 20.7 degrees 2-theta±0.2 degrees 2-theta; and combinations of these data.

Crystalline Form 2 of Gefapixant may be further characterized by an X-ray powder diffraction pattern having peaks at 9.3, 13.2, 14.6, 16.9 and 20.7 degrees 2-theta±0.2 degrees 2-theta, and also having any one, two, three, four or five additional peaks selected from 11.2, 17.3, 17.7, 21.1 and 23.1 degrees 2-theta±0.2 degrees 2-theta.

Crystalline Form 2 of Gefapixant may be alternatively characterized by an X-ray powder diffraction pattern having peaks at 9.3, 11.2, 13.2, 14.6, 16.9, 17.3, 17.7, 20.7, 21.1, and 23.1 degrees 2-theta±0.2 degrees 2-theta.

In one embodiment of the present disclosure, crystalline Form 2 of Gefapixant is isolated.

Crystalline Form 2 of Gefapixant may be hydrate form.

Crystalline Form 2 of Gefapixant may be characterized by each of the above characteristics alone or by all possible combinations, e.g., an XRPD pattern having peaks at 9.3, 13.2, 14.6, 16.9 and 20.7 degrees 2-theta±0.2 degrees 2-theta; an XRPD pattern as depicted in FIG. 2, and combinations thereof.

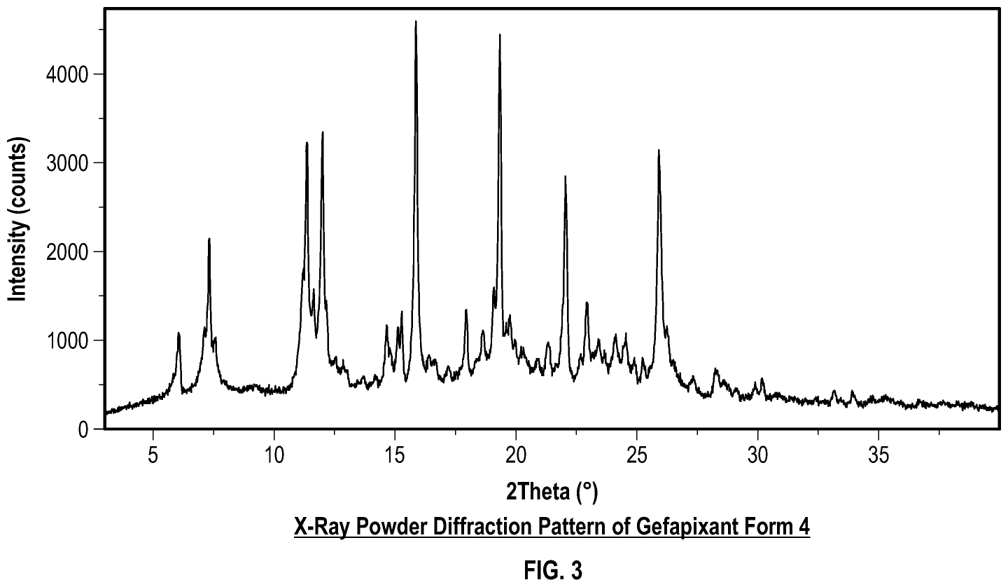

The present disclosure includes a crystalline polymorph of Gefapixant, designated Form 4. The crystalline Form 4 of Gefapixant may be characterized by data selected from one or more of the following: an X-ray powder diffraction pattern substantially as depicted in FIG. 3; an X-ray powder diffraction pattern having peaks at 6.1, 11.4, 15.9, 22.1 and 25.9 degrees 2-theta±0.2 degrees 2-theta; and combinations of these data.

Crystalline Form 4 of Gefapixant may be further characterized by an X-ray powder diffraction pattern having peaks at 6.1, 11.4, 15.9, 22.1 and 25.9 degrees 2-theta±0.2 degrees 2-theta, and also having any one, two, three, four or five additional peaks selected from 7.3, 12.0, 14.7, 19.3 and 22.9 degrees 2-theta±0.2 degrees 2-theta.

Crystalline Form 4 of Gefapixant may be alternatively characterized by an X-ray powder diffraction pattern having peaks at 6.1, 7.3, 11.4, 12.0, 14.7, 15.9, 19.3, 22.9 22.1, and 25.9 degrees 2-theta±0.2 degrees 2-theta.

In one embodiment of the present disclosure, crystalline Form 4 of Gefapixant is isolated.

Crystalline Form 4 of Gefapixant may be acetic acid solvate form.

Crystalline Form 4 of Gefapixant may be characterized by each of the above characteristics alone or by all possible combinations, e.g., an XRPD pattern having peaks at 6.1, 11.4, 15.9, 22.1 and 25.9 degrees 2-theta±0.2 degrees 2-theta; an XRPD pattern as depicted in FIG. 3, and combinations thereof.

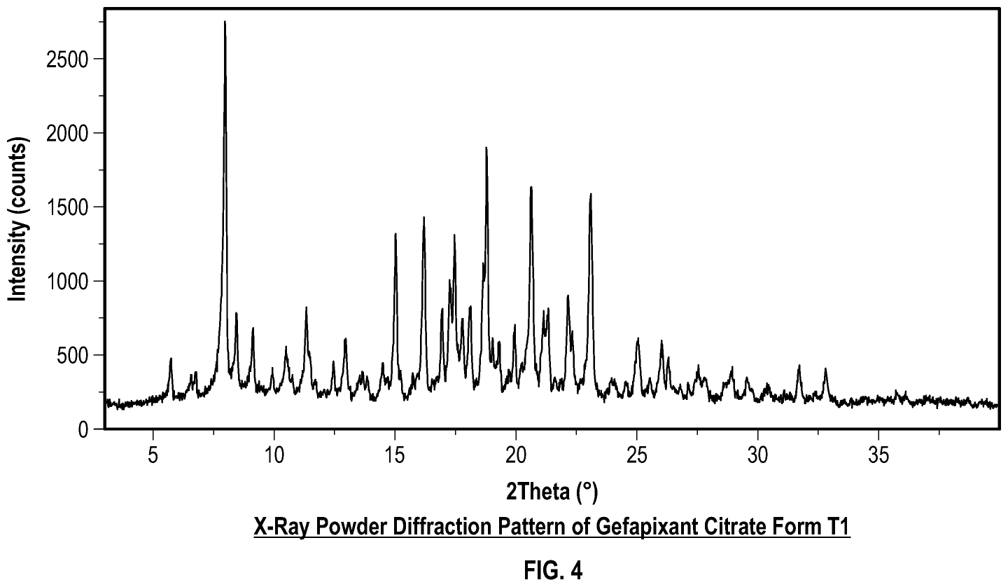

The present disclosure includes a crystalline polymorph of Gefapixant citrate, designated Form T1. The crystalline Form T1 of Gefapixant citrate may be characterized by data selected from one or more of the following: an X-ray powder diffraction pattern substantially as depicted in FIG. 4; an X-ray powder diffraction pattern having peaks at 8.0, 10.5, 15.0, 18.8, and 20.6 degrees 2-theta±0.2 degrees 2-theta; and combinations of these data.

Crystalline Form T1 of Gefapixant citrate may be further characterized by an X-ray powder diffraction pattern having peaks at 8.0, 10.5, 15.0, 18.8, and 20.6 degrees 2-theta±0.2 degrees 2-theta, and also having any one, two, three, four or five additional peaks selected from 8.5, 9.1, 17.5, 18.1 and 20.0 degrees 2-theta±0.2 degrees 2-theta.

Crystalline Form T1 of Gefapixant citrate may be alternatively characterized by an X-ray powder diffraction pattern having peaks at 8.0, 8.5, 9.1, 10.5, 15.0, 17.5, 18.1, 18.8, 20.0,−+ and 20.6 degrees 2-theta±0.2 degrees 2-theta.

In one embodiment of the present disclosure, crystalline Form T1 of Gefapixant citrate is isolated.

Crystalline Form T1 of Gefapixant citrate may be THF solvate form.

Crystalline Form T1 of Gefapixant citrate may be characterized by each of the above characteristics alone or by all possible combinations, e.g., an XRPD pattern having peaks at 8.0, 10.5, 15.0, 18.8, and 20.6 degrees 2-theta±0.2 degrees 2-theta; an XRPD pattern as depicted in FIG. 4, and combinations thereof.

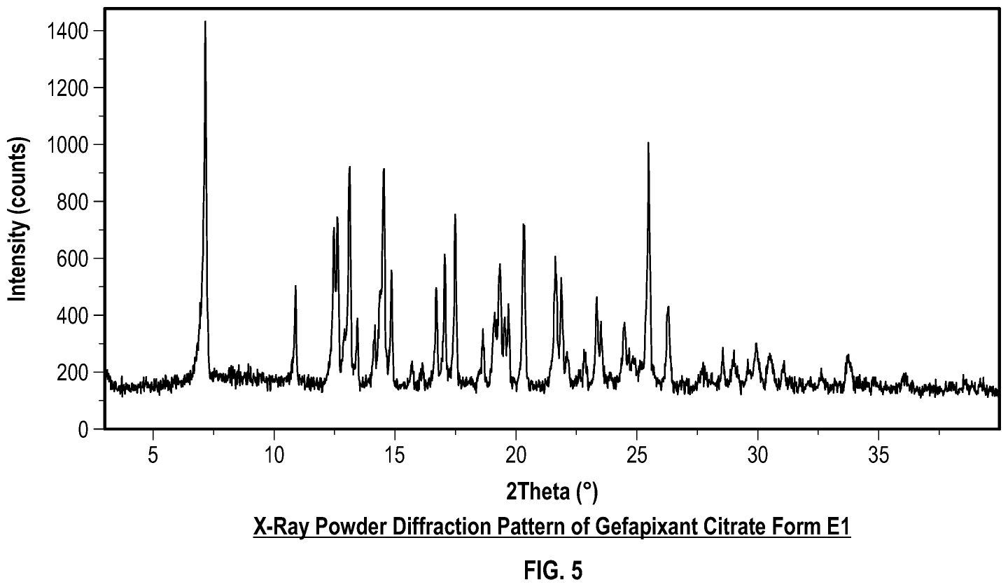

The present disclosure includes a crystalline polymorph of Gefapixant citrate, designated Form E1. The crystalline Form E1 of Gefapixant citrate may be characterized by data selected from one or more of the following: an X-ray powder diffraction pattern substantially as depicted in FIG. 5; an X-ray powder diffraction pattern having peaks at 7.2, 10.9, 13.1, 14.5 and 14.9 degrees 2-theta±0.2 degrees 2-theta; and combinations of these data.

Crystalline Form E1 of Gefapixant citrate may be further characterized by an X-ray powder diffraction pattern having peaks at 7.2, 10.9, 13.1, 14.5 and 14.9 degrees 2-theta±0.2 degrees 2-theta, and also having any one, two, three, or four additional peaks selected from 16.8, 17.1, 17.5 and 25.5 degrees 2-theta±0.2 degrees 2-theta.

Crystalline Form E1 of Gefapixant citrate may be alternatively characterized by an X-ray powder diffraction pattern having peaks at 7.2, 10.9, 13.1, 14.5, 14.9, 16.8, 17.1, 17.5, and 25.5 degrees 2-theta±0.2 degrees 2-theta.

In one embodiment of the present disclosure, crystalline Form E1 of Gefapixant citrate is isolated.

Crystalline Form E1 of Gefapixant citrate may be ethanol solvate form.

Crystalline Form E1 of Gefapixant citrate may be characterized by each of the above characteristics alone or by all possible combinations, e.g., an XRPD pattern having peaks at 7.2, 10.9, 13.1, 14.5 and 14.9 degrees 2-theta±0.2 degrees 2-theta; an XRPD pattern as depicted in FIG. 5, and combinations thereof.

The present disclosure includes Gefapixant maleate, typically it is in a crystalline form.

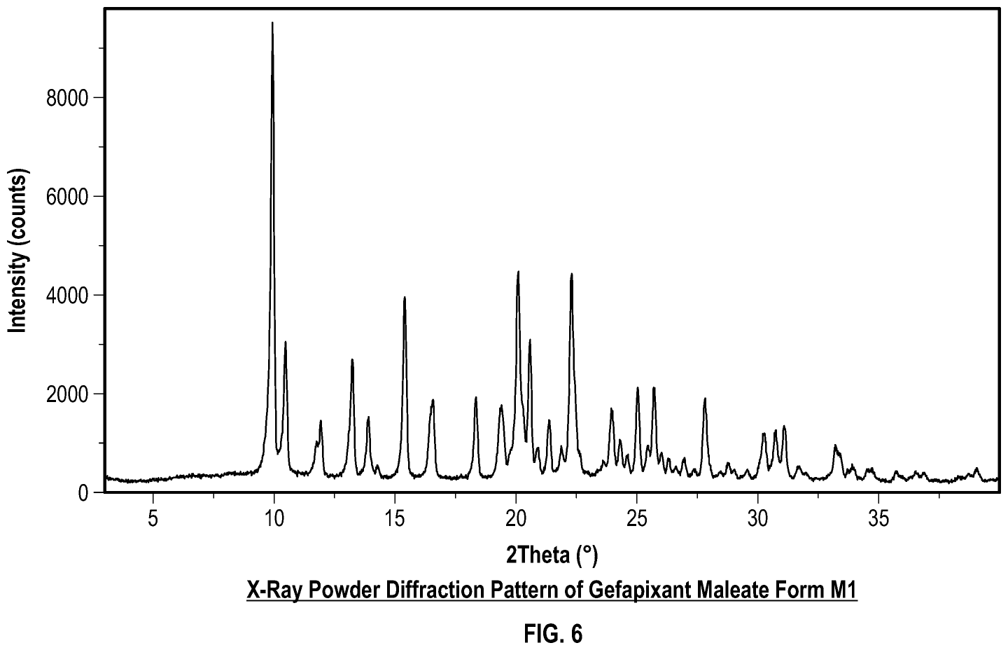

The present disclosure includes a crystalline polymorph of Gefapixant maleate, designated Form M1. The crystalline Form M1 of Gefapixant maleate may be characterized by data selected from one or more of the following: an X-ray powder diffraction pattern substantially as depicted in FIG. 6; an X-ray powder diffraction pattern having peaks at 10.0, 13.2, 15.5, 18.4 and 22.3 degrees 2-theta±0.2 degrees 2-theta; and combinations of these data.

Crystalline Form M1 of Gefapixant maleate may be further characterized by an X-ray powder diffraction pattern having peaks at 10.0, 13.2, 15.5, 18.4 and 22.3 degrees 2-theta±0.2 degrees 2-theta, and also having any one, two, three, four or five additional peaks selected from 10.5, 16.6, 20.1, 21.4 and 24.0 degrees 2-theta±0.2 degrees 2-theta.

Crystalline Form M1 of Gefapixant maleate may be alternatively characterized by an X-ray powder diffraction pattern having peaks at 10.0, 10.5, 13.2, 15.5, 16.6, 18.4, 20.1, 21.4, 22.3, and 24.0 degrees 2-theta±0.2 degrees 2-theta.

Crystalline Form M1 of Gefapixant maleate may be alternatively characterized by the peaks presented in Table 1 below (left column), either with or without the indicated relative intensity values:

TABLE 1

| Pos. [°2Th.]/±0.2° 2θ | Rel. Int. [%] |
|---|---|
| 10.0 | 100 |
| 10.5 | 29 |
| 11.8 | 8 |
| 11.9 | 13 |
| 13.2 | 26 |
| 13.9 | 13 |
| 15.5 | 39 |
| 16.4 | 12 |
| 16.6 | 18 |
| 18.4 | 18 |
| 19.3 | 15 |
| 19.5 | 12 |
| 20.1 | 45 |
| 20.6 | 28 |
| 20.9 | 7 |
| 21.4 | 13 |
| 21.9 | 7 |
| 22.3 | 45 |
| 22.5 | 18 |
| 24.0 | 16 |
| 24.3 | 9 |
| 24.6 | 6 |
| 25.0 | 20 |
| 25.5 | 8 |
| 25.7 | 20 |
| 26.0 | 6 |
| 26.3 | 5 |
| 26.4 | 5 |
| 27.0 | 5 |
| 27.8 | 18 |
| 30.3 | 10 |
| 30.7 | 11 |
| 31.1 | 11 |
| 33.2 | 7 |
| 33.4 | 5 |

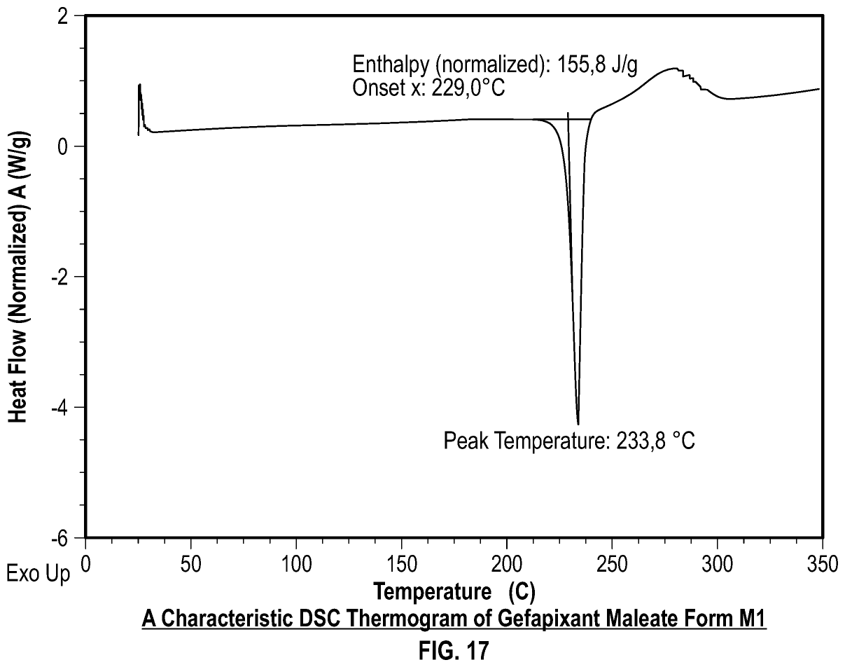

Alternatively, or additionally, crystalline Form M1 of Gefapixant maleate may be further characterized by a DSC melting onset at a temperature of about 229.0° C. or by a DSC thermogram as depicted in FIG. 17. Typically, the DSC onset melting temperature may be at a range of: about 226.0° C. to about 232.0° C., about 226.0° C. to about 231.0° C., about 227.0° C. to about 232.0° C., about 227.0° C. to about 231.0° C., or about 229.0° C.±1.0° C. In one embodiment of the present disclosure, crystalline Form M1 of Gefapixant maleate is isolated.

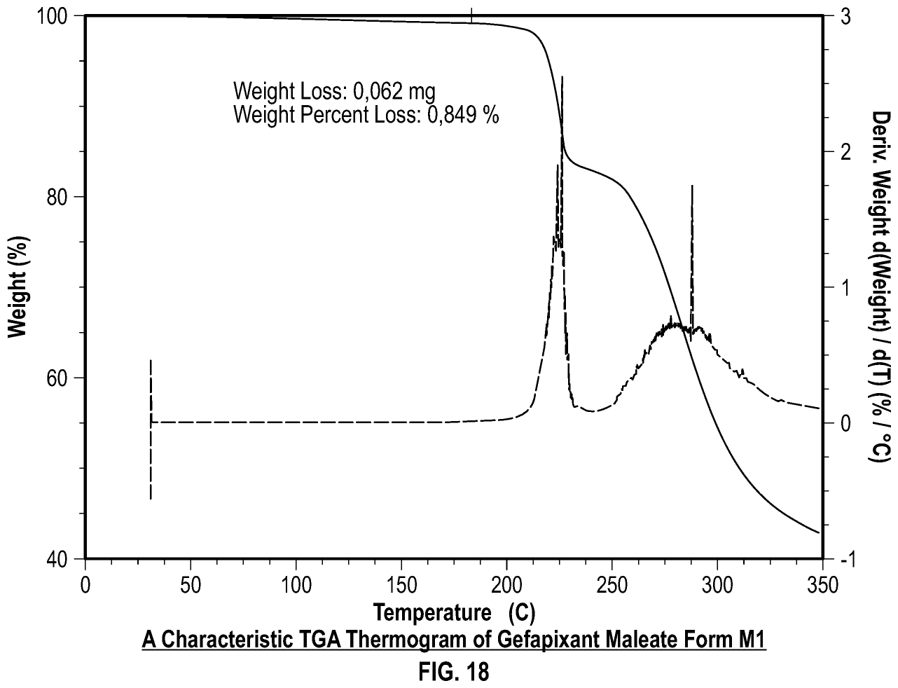

Crystalline Form M1 of Gefapixant maleate may be anhydrous form. A typical TGA is presented in FIG. 18.

Crystalline form M1 of Gefapixant maleate may be a 1:1 salt.

Crystalline Form M1 of Gefapixant maleate may be characterized by each of the above characteristics alone or by all possible combinations, e.g., an XRPD pattern having peaks at 10.0, 13.2, 15.5, 18.4 and 22.3 degrees 2-theta±0.2 degrees 2-theta; an XRPD pattern as depicted in FIG. 6, and combinations thereof.

The present disclosure includes Gefapixant camsylate; typically it is in a crystalline form.

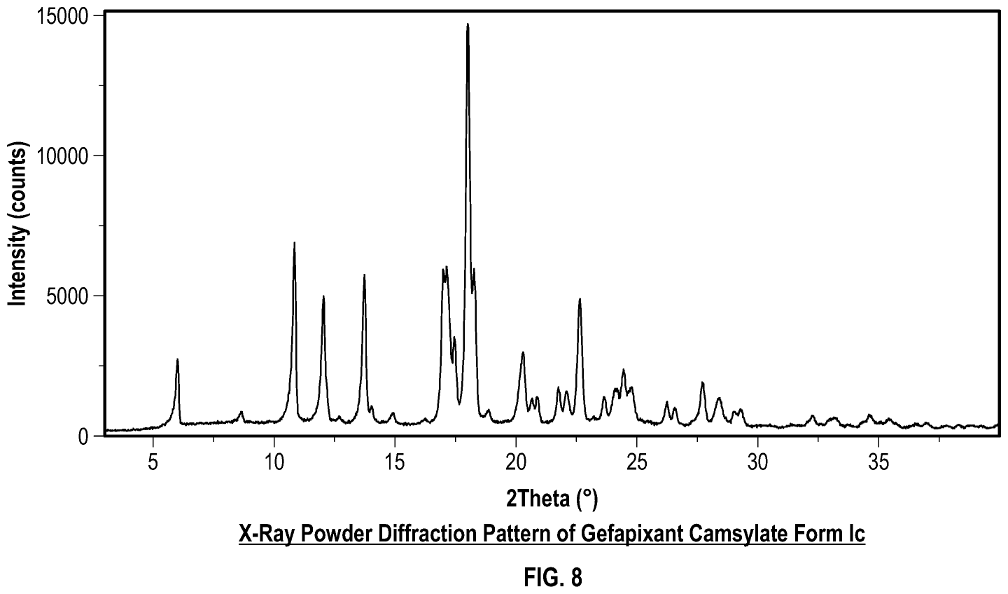

The present disclosure includes a crystalline polymorph of Gefapixant camsylate, designated Form Ic. The crystalline Form Ic of Gefapixant camsylate may be characterized by data selected from one or more of the following: an X-ray powder diffraction pattern substantially as depicted in FIG. 8; an X-ray powder diffraction pattern having peaks at 6.0, 18.0, 22.6 and 24.4 degrees 2-theta±0.2 degrees 2-theta; and combinations of these data.

Crystalline Form Ic of Gefapixant camsylate may be further characterized by an X-ray powder diffraction pattern having peaks at 6.0, 18.0, 22.6 and 24.4 degrees 2-theta±0.2 degrees 2-theta, and also having any one, two, three, or four additional peaks selected from 10.9, 21.8, 24.9 and 22.1 degrees 2-theta±0.2 degrees 2-theta.

Crystalline Form Ic of Gefapixant camsylate may be alternatively characterized by an X-ray powder diffraction pattern having peaks at 6.0, 10.9, 18.0, 21.8, 22.1, 22.6, 24.9, and 24.4 degrees 2-theta±0.2 degrees 2-theta.

Crystalline Form Ic of Gefapixant camsylate may be alternatively characterized by the peaks presented in Table 2 below (left column), either with or without the indicated relative intensity values:

GFP camsylate form Ic

| Pos. [°2Th.]/±0.2° 2θ | Rel. Int. [%] |
|---|---|
| 6.0 | 16 |
| 10.9 | 23 |
| 12.0 | 25 |
| 12.7 | 2 |
| 13.7 | 26 |
| 17.0 | 29 |
| 17.1 | 24 |
| 17.3 | 18 |
| 17.5 | 15 |
| 18.0 | 100 |
| 18.3 | 22 |
| 20.3 | 12 |
| 20.7 | 5 |
| 20.9 | 5 |
| 21.8 | 6 |
| 22.1 | 6 |
| 22.6 | 28 |
| 23.6 | 5 |
| 24.0 | 7 |
| 24.4 | 9 |
| 24.9 | 6 |
| 27.6 | 6 |
| 27.7 | 6 |
| 28.3 | 5 |

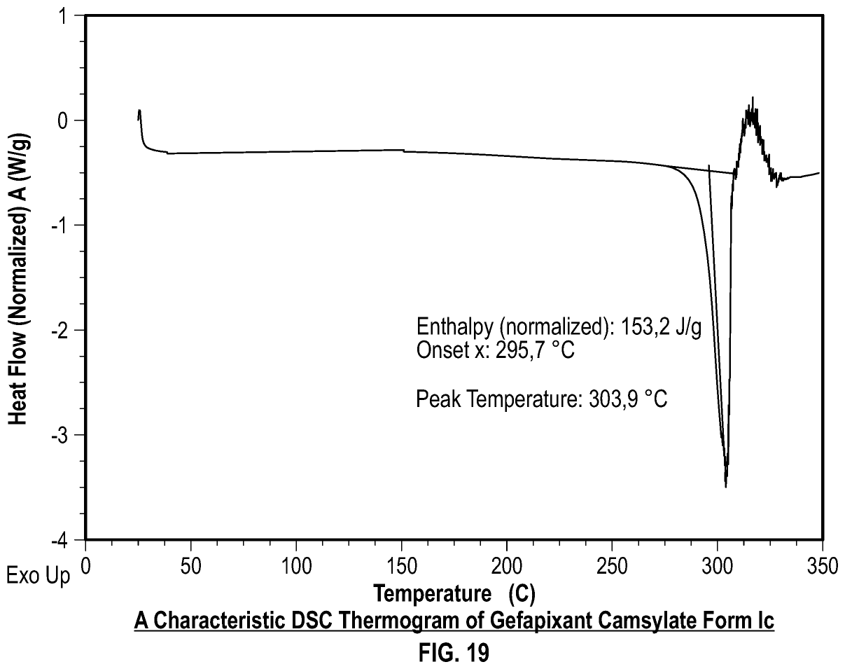

Alternatively, or additionally, crystalline Form Ic of Gefapixant camsylate may be further characterized by a DSC melting onset melting at a temperature of about 295.7° C. or by a DSC thermogram as depicted in FIG. 19. Typically, the DSC onset melting temperature may be at a range of: about 292.7° C. to about 298.7° C., about 292.7° C. to about 297.7° C., about 293.7° C. to about 298.7° C., about 293.7° C. to about 297.7° C., or 295.7° C.±1.0° C.

In one embodiment of the present disclosure, crystalline Form Ic of Gefapixant camsylate is isolated.

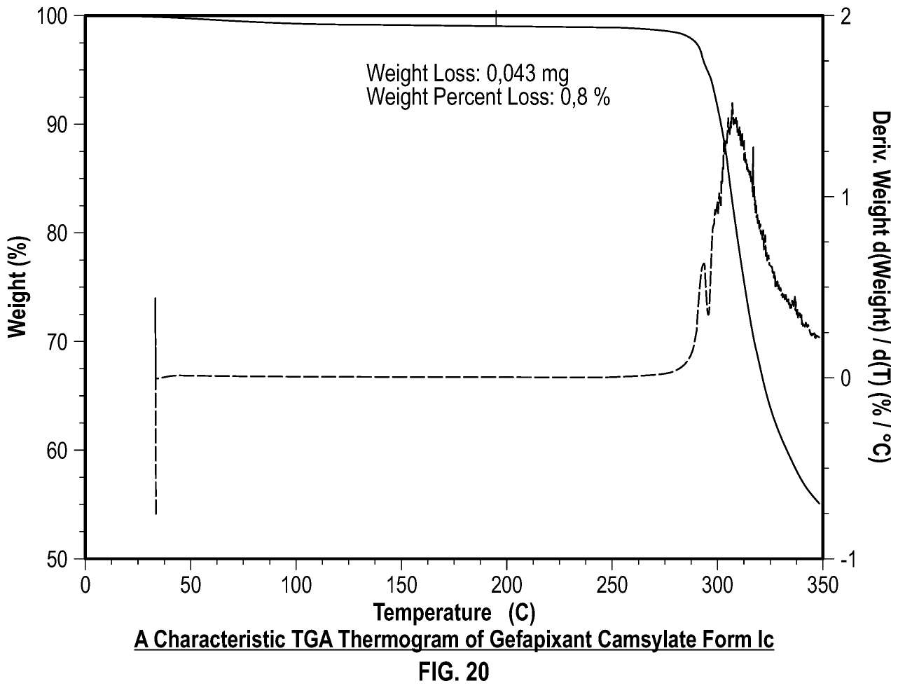

Crystalline Form Ic of Gefapixant camsylate may be anhydrous form. A typical TGA is presented in FIG. 20.

Crystalline form Ic of Gefapixant maleate may be a 1:1 salt.

Crystalline Form Ic of Gefapixant camsylate may be characterized by each of the above characteristics alone or by all possible combinations, e.g., an XRPD pattern having peaks at 6.0, 18.0, 22.6 and 24.4 degrees 2-theta±0.2 degrees 2-theta; an XRPD pattern as depicted in FIG. 8, and combinations thereof.

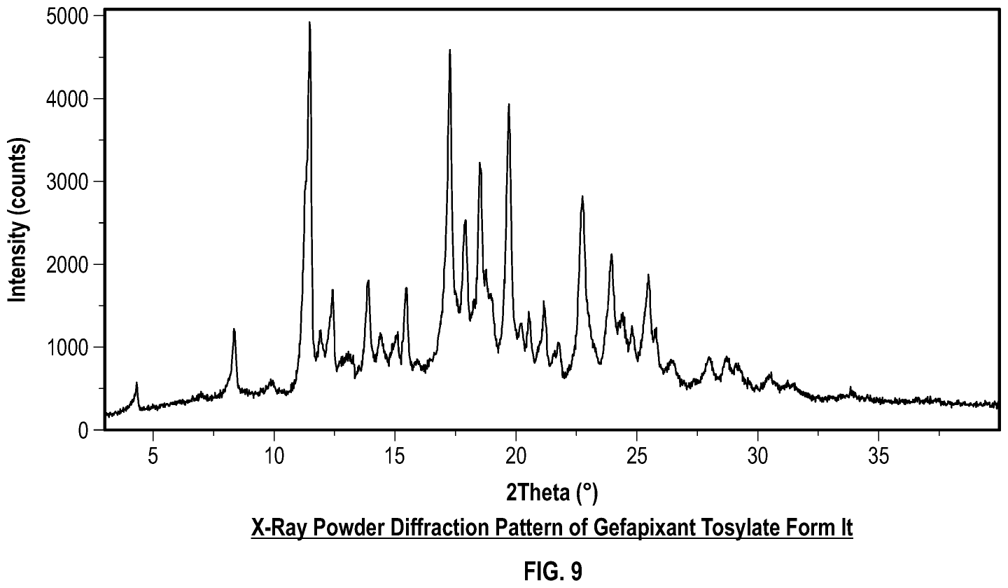

The present disclosure includes a crystalline polymorph of Gefapixant tosylate, designated Form It. The crystalline Form It of Gefapixant tosylate may be characterized by data selected from one or more of the following: an X-ray powder diffraction pattern substantially as depicted in FIG. 9; an X-ray powder diffraction pattern having peaks at 8.4, 11.5, 15.5, 19.7 and 22.8 degrees 2-theta±0.2 degrees 2-theta; and combinations of these data.

Crystalline Form It of Gefapixant tosylate may be further characterized by an X-ray powder diffraction pattern having peaks at 8.4, 11.5, 15.5, 19.7 and 22.8 degrees 2-theta±0.2 degrees 2-theta, and also having any one, two, three, or four additional peaks selected from 12.4, 13.9, 17.9 and 21.2 degrees 2-theta±0.2 degrees 2-theta.

Crystalline Form It of Gefapixant tosylate may be alternatively characterized by an X-ray powder diffraction pattern having peaks at 8.4, 11.5, 12.4, 13.9, 15.5, 17.9, 19.7, 21.2, and 22.8 degrees 2-theta±0.2 degrees 2-theta.

In one embodiment of the present disclosure, crystalline Form It of Gefapixant tosylate is isolated.

Crystalline Form It of Gefapixant tosylate may be characterized by each of the above characteristics alone or by all possible combinations, e.g., an XRPD pattern having peaks at 8.4, 11.5, 15.5, 19.7 and 22.8 degrees 2-theta±0.2 degrees 2-theta; an XRPD pattern as depicted in FIG. 9, and combinations thereof.

The present disclosure includes Gefapixant hemiedisylate, typically it is in a crystalline form.

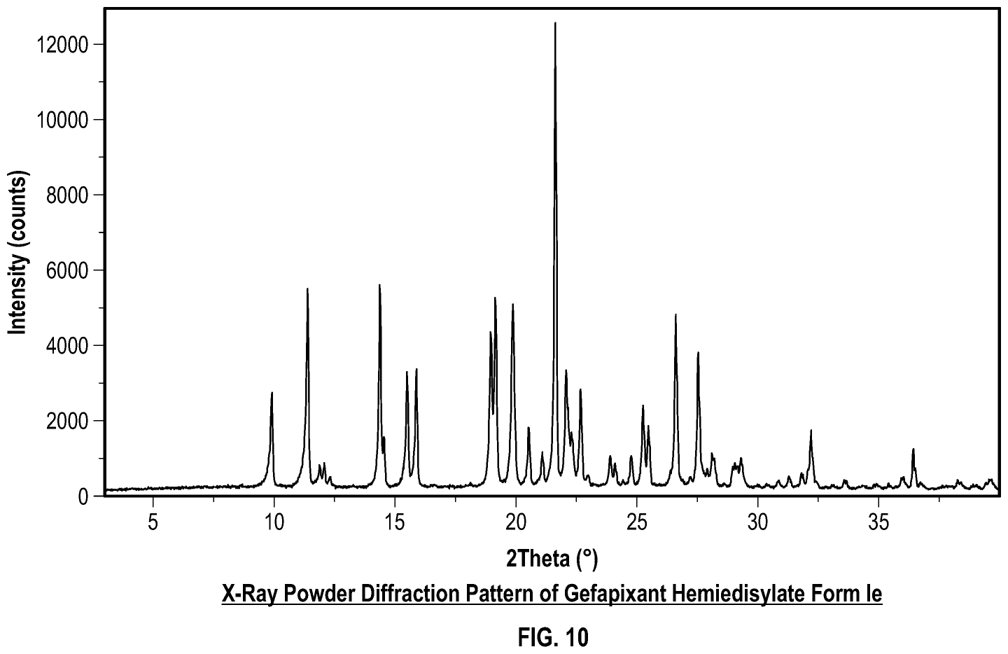

The present disclosure includes a crystalline polymorph of Gefapixant hemiedisylate, designated Form Ie. The crystalline Form Ie of Gefapixant hemiedisylate may be characterized by data selected from one or more of the following: an X-ray powder diffraction pattern substantially as depicted in FIG. 10; an X-ray powder diffraction pattern having peaks at 14.4, 15.9, 19.9, 21.6, and 27.6 degrees 2-theta±0.2 degrees 2-theta; and combinations of these data.

Crystalline Form Ie of Gefapixant hemiedisylate may be further characterized by an X-ray powder diffraction pattern having peaks at 14.4, 15.9, 19.9, 21.6, and 27.6 degrees 2-theta±0.2 degrees 2-theta, and also having any one, two, three, four or five additional peaks selected from 11.4, 19.2, 21.1, 22.7 and 28.1 degrees 2-theta±0.2 degrees 2-theta.

Crystalline Form 1e of Gefapixant hemiedisylate may be alternatively characterized by an X-ray powder diffraction pattern having peaks at 11.4, 14.4, 15.9, 19.2, 19.9, 21.1, 21.6, 22.7, 27.6, and 28.1 degrees 2-theta±0.2 degrees 2-theta.

Crystalline Form 1e of Gefapixant hemiedisylate may be alternatively characterized by the peaks presented in Table 3 below (left column), either with or without the indicated relative intensity values

TABLE 3
GFP hemiedisylate form Ie

| Pos. [°2Th.]/±0.2° 2θ | Rel. Int. [%] |
|---|---|
| 9.9 | 20 |
| 11.4 | 41 |
| 11.9 | 5 |
| 12.1 | 5 |
| 14.4 | 43 |
| 14.6 | 10 |
| 15.5 | 23 |
| 15.9 | 25 |
| 19.0 | 34 |
| 19.2 | 40 |
| 19.9 | 40 |
| 20.5 | 13 |
| 21.1 | 7 |
| 21.6 | 100 |
| 22.1 | 25 |
| 22.3 | 11 |
| 22.7 | 21 |
| 23.9 | 7 |
| 24.1 | 5 |
| 24.8 | 7 |
| 25.2 | 17 |
| 25.5 | 13 |
| 26.6 | 38 |
| 27.6 | 29 |
| 28.1 | 7 |
| 29.3 | 6 |
| 32.2 | 12 |
| 36.4 | 8 |

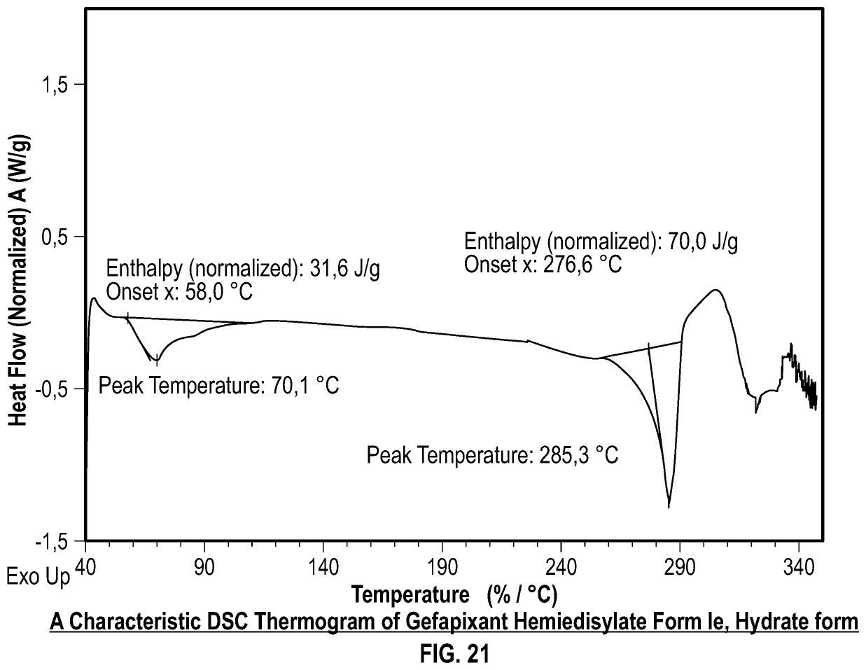

Alternatively or additionally, crystalline Form Ie of Gefapixant hemiedisylate may be further characterized by a DSC onset at a temperature of about 276.6° C. or by a DSC thermogram as depicted in FIG. 21. Typically, the DSC onset temperature may be at a range of: about 273.6° C. to about 279.6° C., about 273.6° C. to about 278.6° C., about 274.6° C. to about 279.6° C., about 274.6° C. to about 278.6° C., or 276.6° C.±1.0° C.

Preferably crystalline Form Ie according to this embodiment is a hydrate form.

In one embodiment of the present disclosure, crystalline Form Ie of Gefapixant hemiedisylate is isolated.

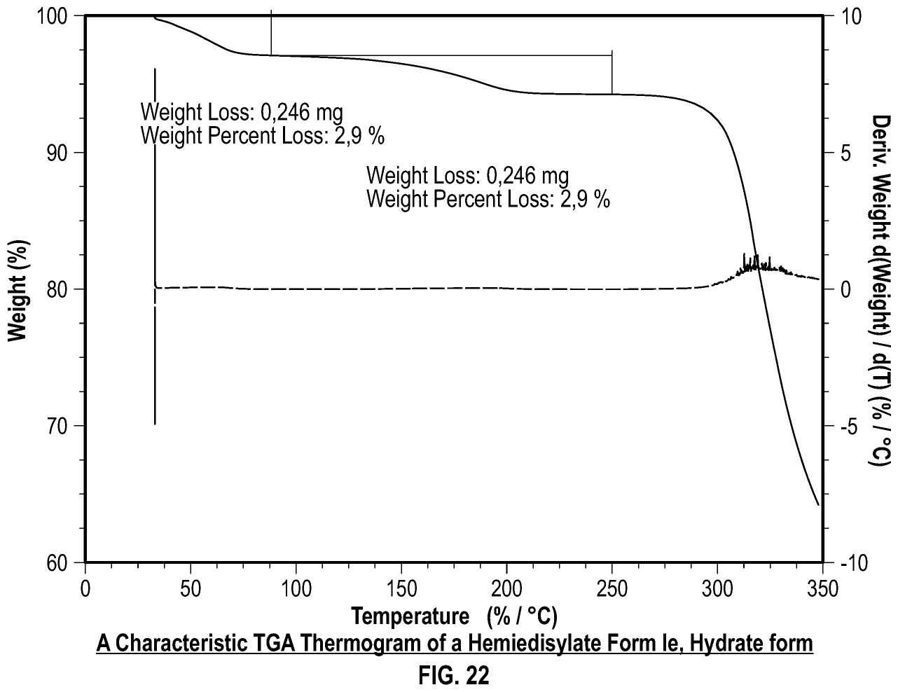

As indicated above, crystalline Form Ie of Gefapixant hemiedisylate may be a hydrate form. A typical TGA is presented in FIG. 22.

Crystalline Form Ie of Gefapixant hemiedisylate may be characterized by each of the above characteristics alone or by all possible combinations, e.g., an XRPD pattern having peaks at 14.4, 15.9, 19.9, 21.6 and 27.6 degrees 2-theta±0.2 degrees 2-theta; an XRPD pattern as depicted in FIG. 10, and combinations thereof.

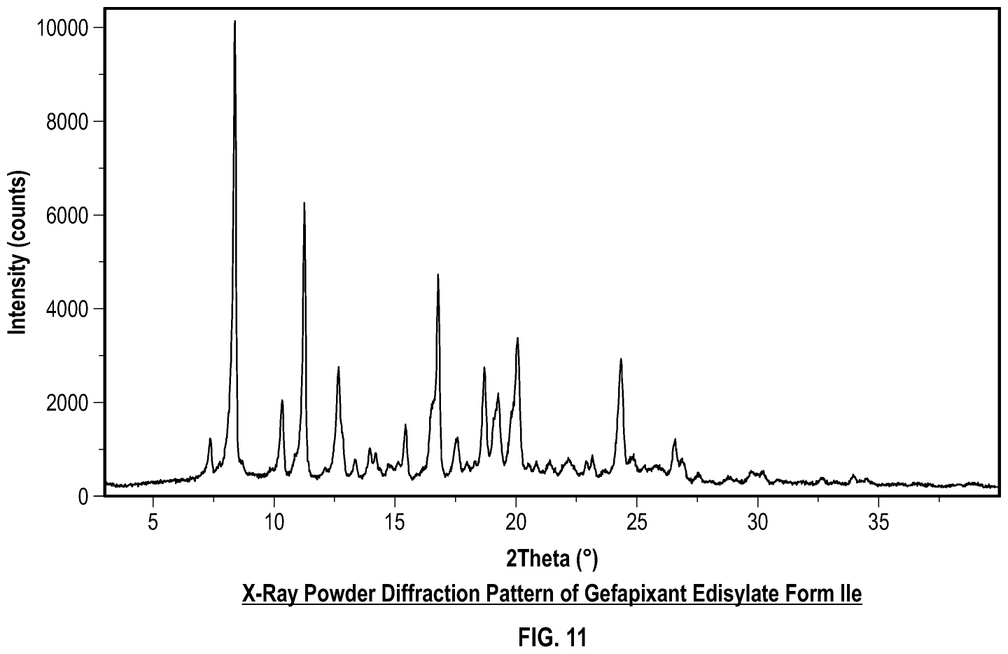

The present disclosure includes a crystalline polymorph of Gefapixant edisylate, designated Form IIe. The crystalline Form IIe of Gefapixant edisylate may be characterized by data selected from one or more of the following: an X-ray powder diffraction pattern substantially as depicted in FIG. 11; an X-ray powder diffraction pattern having peaks at 7.4, 8.4, 12.7, and 16.8 degrees 2-theta±0.2 degrees 2-theta; and combinations of these data.

Crystalline Form IIe of Gefapixant edisylate may be further characterized by an X-ray powder diffraction pattern having peaks at 7.4, 8.4, 12.7 and 16.8 degrees 2-theta±0.2 degrees 2-theta, and having any one, two, three, or four additional peaks selected from 10.4, 11.3, 13.4 and 20.1 degrees 2-theta±0.2 degrees 2-theta.

Crystalline Form IIe of Gefapixant edisylate may be alternatively characterized by an X-ray powder diffraction pattern having peaks at 7.4, 8.4, 10.4, 11.3, 12.7, 13.4, 16.8, and 20.1 degrees 2-theta±0.2 degrees 2-theta.

In one embodiment of the present disclosure, crystalline Form IIe of Gefapixant edisylate is isolated.

Crystalline Form IIe of Gefapixant edisylate may be hydrate form.

Crystalline Form IIe of Gefapixant edisylate may be characterized by each of the above characteristics alone or by all possible combinations, e.g., an XRPD pattern having peaks at 7.4, 8.4, 12.7, 16.8 degrees 2-theta±0.2 degrees 2-theta; an XRPD pattern as depicted in FIG. 11, and combinations thereof.

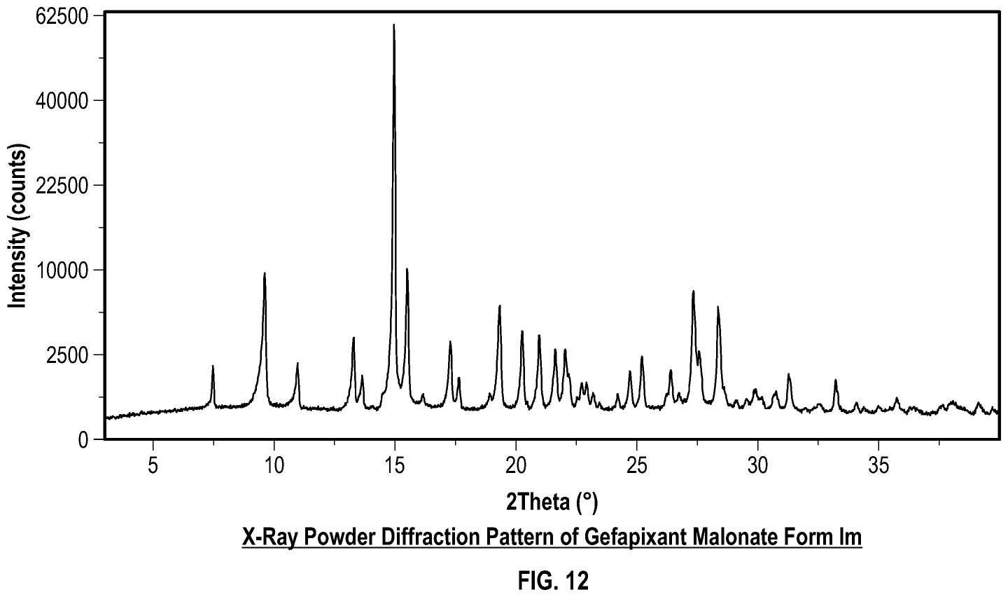

The present disclosure includes a crystalline polymorph of Gefapixant malonate, designated Form Im. The crystalline Form Im of Gefapixant malonate may be characterized by data selected from one or more of the following: an X-ray powder diffraction pattern substantially as depicted in FIG. 12; an X-ray powder diffraction pattern having peaks at 9.6, 15.0, 15.5, 22.0 and 28.4 degrees 2-theta±0.2 degrees 2-theta; and combinations of these data.

Crystalline Form Im of Gefapixant malonate may be further characterized by an X-ray powder diffraction pattern having peaks at 9.6, 15.0, 15.5, 22.0 and 28.4 degrees 2-theta±0.2 degrees 2-theta, and also having any one, two, three, or four additional peaks selected from 13.3, 21.0, 21.6 and 24.7 degrees 2-theta±0.2 degrees 2-theta.

Crystalline Form Im of Gefapixant malonate may be alternatively characterized by an X-ray powder diffraction pattern having peaks at 9.6, 13.3, 15.0, 15.5, 21.0, 21.6, 22.0, 24.7, and 28.4 degrees 2-theta±0.2 degrees 2-theta.

In one embodiment of the present disclosure, crystalline Form Im of Gefapixant malonate is isolated.

Crystalline Form Im of Gefapixant malonate may be anhydrous form.

Crystalline Form Im of Gefapixant malonate may be characterized by each of the above characteristics alone or by all possible combinations, e.g., an XRPD pattern having peaks at 9.6, 15.0, 15.5, 22.0 and 28.4 degrees 2-theta±0.2 degrees 2-theta; an XRPD pattern as depicted in FIG. 12, and combinations thereof.

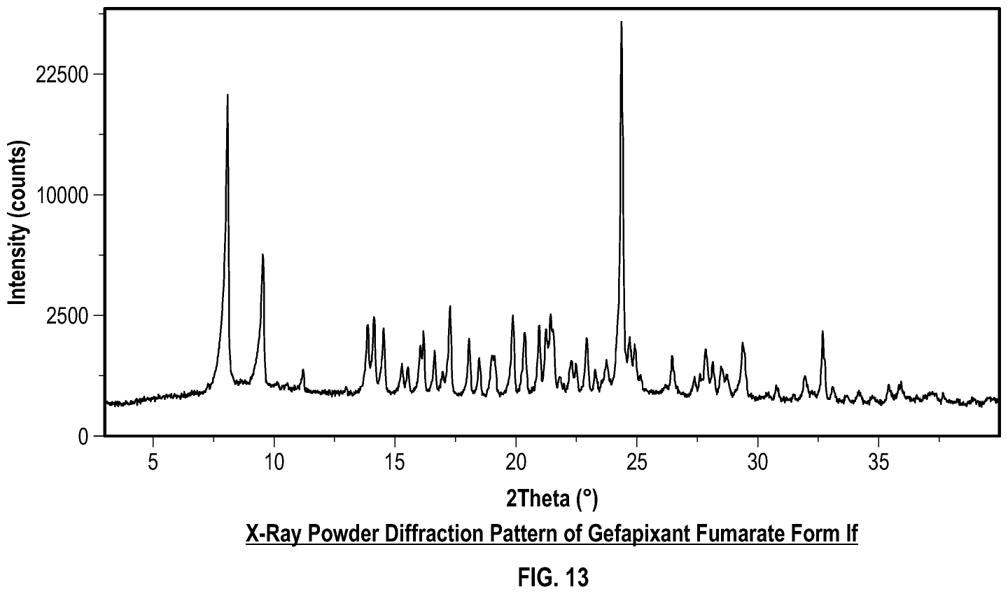

The present disclosure includes a crystalline polymorph of Gefapixant fumarate, designated Form If. The crystalline Form If of Gefapixant fumarate may be characterized by data selected from one or more of the following: an X-ray powder diffraction pattern substantially as depicted in FIG. 13; an X-ray powder diffraction pattern having peaks at 8.1, 9.6, 14.5, 19.9 and 24.4 degrees 2-theta±0.2 degrees 2-theta; and combinations of these data.

Crystalline Form If of Gefapixant fumarate may be further characterized by an X-ray powder diffraction pattern having peaks at 8.1, 9.6, 14.5, 19.9 and 24.4 degrees 2-theta±0.2 degrees 2-theta, and also having any one, two, three, or four additional peaks selected from 13.9, 18.1, 19.1 and 20.4 degrees 2-theta±0.2 degrees 2-theta.

Crystalline Form If of Gefapixant fumarate may be alternatively characterized by an X-ray powder diffraction pattern having peaks at 8.1, 9.6, 13.9, 14.5, 18.1, 19.1 19.9, 20.4, and 24.4 degrees 2-theta±0.2 degrees 2-theta.

In one embodiment of the present disclosure, crystalline Form If of Gefapixant fumarate is isolated.

Crystalline Form If of Gefapixant fumarate may be characterized by each of the above characteristics alone or by all possible combinations, e.g., an XRPD pattern having peaks at 8.1, 9.6, 14.5, 19.9 and 24.4 degrees 2-theta±0.2 degrees 2-theta; an XRPD pattern as depicted in FIG. 13, and combinations thereof.

The present disclosure further encompasses a crystalline complex of Gefapixant citrate and L-Lysine. Crystalline Gefapixant citrate: L-Lysine complexes may be a co-crystal of Gefapixant citrate and L-Lysine.

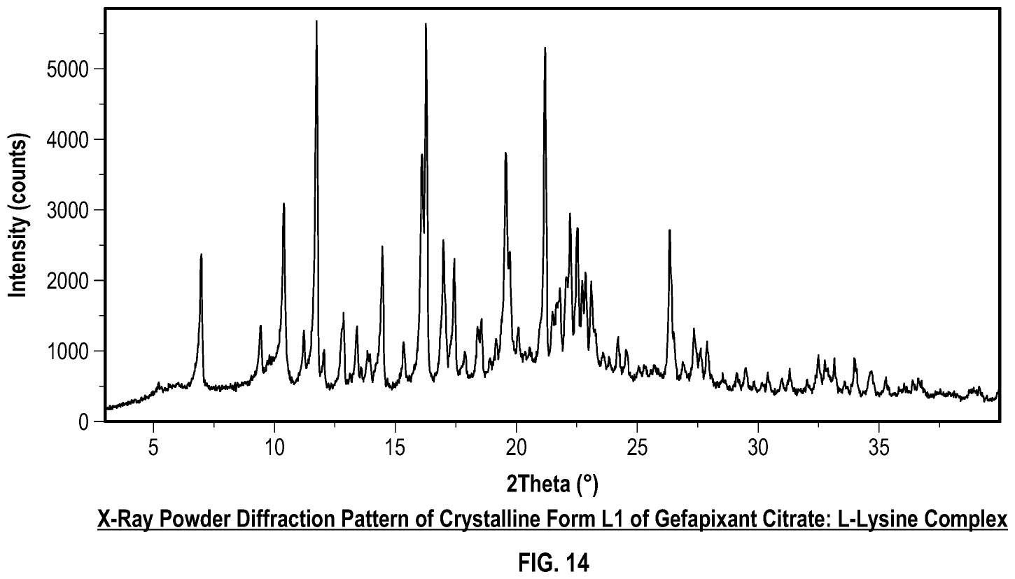

The present disclosure includes a crystalline complex of Gefapixant citrate and L-Lysine, designated Form L1. Crystalline Form L1 of Gefapixant citrate: L-Lysine complex may be characterized by data selected from one or more of the following: an X-ray powder diffraction pattern substantially as depicted in FIG. 14; an X-ray powder diffraction pattern having peaks at 7.0, 11.8, 14.5, 21.2 and 26.3 degrees 2-theta±0.2 degrees 2-theta; and combinations of these data.

Crystalline Form L1 of Gefapixant citrate: L-Lysine complex may be further characterized by an X-ray powder diffraction pattern having peaks at 7.0, 11.8, 14.5, 21.2 and 26.3 degrees 2-theta±0.2 degrees 2-theta, and also having any one, two, three, four and five additional peaks selected from 12.9, 13.4, 17.5, 23.1 and 24.6 degrees 2-theta±0.2 degrees 2-theta.

Crystalline Form L1 of Gefapixant citrate: L-Lysine complex may be alternatively characterized by an X-ray powder diffraction pattern having peaks at 7.0, 11.8, 12.9, 13.4, 14.5, 17.5, 21.2, 23.1, 24.6, and 26.3 degrees 2-theta±0.2 degrees 2-theta.

In one embodiment of the present disclosure, crystalline Form L1 of Gefapixant citrate: L-Lysine complex is isolated.

In one embodiment of the present disclosure, Crystalline Form L1 of Gefapixant citrate: L-Lysine complex may be anhydrous form.

Crystalline Form L1 of Gefapixant citrate: L-Lysine complex may be characterized by each of the above characteristics alone or by all possible combinations, e.g., an XRPD pattern having peaks at 7.0, 11.8, 14.5, 21.2 and 26.3 degrees 2-theta±0.2 degrees 2-theta; an XRPD pattern as depicted in FIG. 14, and combinations thereof.

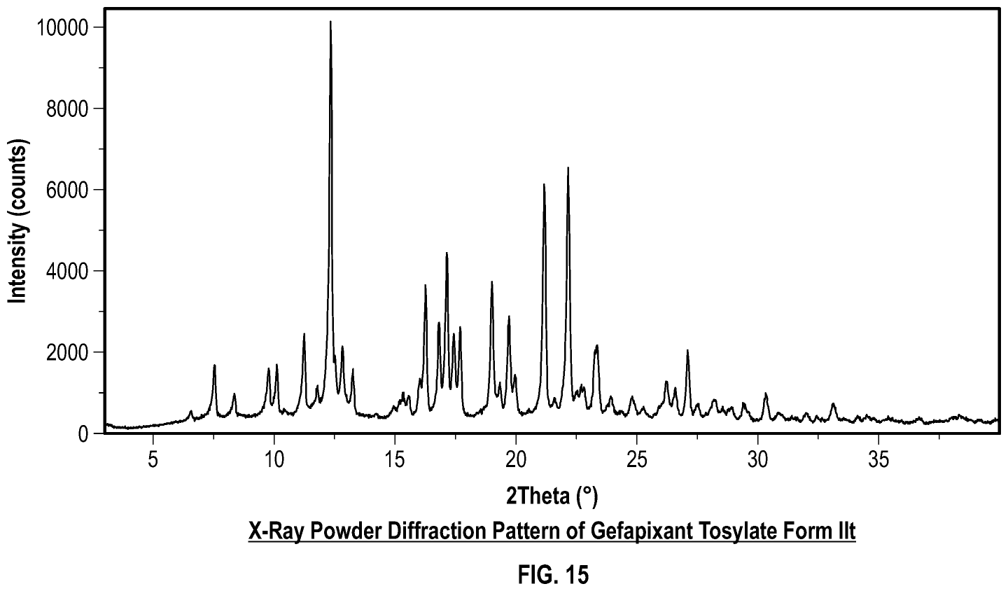

The present disclosure includes a crystalline polymorph of Gefapixant tosylate, designated Form M. The crystalline Form IIt of Gefapixant tosylate may be characterized by data selected from one or more of the following: an X-ray powder diffraction pattern substantially as depicted in FIG. 15; an X-ray powder diffraction pattern having peaks at 7.6, 12.4, 19.0, 21.2 and 22.2 degrees 2-theta±0.2 degrees 2-theta; and combinations of these data.

Crystalline Form IIt of Gefapixant tosylate may be further characterized by an X-ray powder diffraction pattern having peaks at 7.6, 12.4, 19.0, 21.2 and 22.2 degrees 2-theta±0.2 degrees 2-theta, and also having any one, two, three, four, or five additional peaks selected from 10.2, 12.9, 16.8, 23.4 and 27.1 degrees 2-theta±0.2 degrees 2-theta.

Crystalline Form IIt of Gefapixant tosylate may be alternatively characterized by an X-ray powder diffraction pattern having peaks at 7.6, 10.2, 12.4, 12.9, 16.8, 19.0, 21.2, 22.2, 23.4 and 27.1 degrees 2-theta±0.2 degrees 2-theta.

In one embodiment of the present disclosure, crystalline Form IIt of Gefapixant tosylate is isolated.

Crystalline Form IIt of Gefapixant tosylate may be anhydrous form.

Crystalline Form IIt of Gefapixant tosylate may be characterized by each of the above characteristics alone or by all possible combinations, e.g., an XRPD pattern having peaks at 7.6, 12.4, 19.0, 21.2 and 22.2 degrees 2-theta±0.2 degrees 2-theta; an XRPD pattern as depicted in FIG. 15, and combinations thereof.

The present disclosure includes a crystalline polymorph of Gefapixant fumarate designated Form IIf. The crystalline Form IIf of Gefapixant fumarate may be characterized by an X-ray powder diffraction pattern having peaks at 8.6, 9.9, 17.2 and 25.9 degrees 2-theta±0.2 degrees 2-theta.

Crystalline Form IIf of Gefapixant fumarate may be further characterized by an X-ray powder diffraction pattern having peaks at 8.6, 9.9, 17.2 and 25.9 degrees 2-theta±0.2 degrees 2-theta, and also having any one, two, or three additional peaks selected from 13.7, 16.2 and 20.0 degrees 2-theta±0.2 degrees 2-theta.

Crystalline Form IIf of Gefapixant fumarate may be alternatively characterized by an X-ray powder diffraction pattern having peaks at 8.6, 9.9, 13.7, 16.2, 17.2, 20.0, and 25.9 degrees 2-theta±0.2 degrees 2-theta.

In one embodiment of the present disclosure, crystalline Form IIf of Gefapixant tosylate is isolated.

Crystalline Form IIf of Gefapixant fumarate may be anhydrous form.

Crystalline Form IIf of Gefapixant fumarate may be characterized by each of the above characteristics alone or by all possible combinations, e.g., an XRPD pattern having peaks at 8.6, 9.9, 17.2 and 25.9 degrees 2-theta±0.2 degrees 2-theta.

In any aspect or embodiment of the present disclosure, any of the solid state forms of Gefapixant, Gefapixant salts, or Gefapixant cocrystals, described herein may be polymorphically pure or may be substantially free of any other solid state forms of the subject Gefapixant, Gefapixant salt, or Gefapixant cocrystal (for example a crystalline Gefapixant citrate, which is polymorphically pure, may be substantially free of any other solid state forms of Gefapixant citrate). In any aspect or embodiment of the present disclosure, any of the solid state forms of Gefapixant, Gefapixant salts, or Gefapixant cocrystals described in any aspect or embodiment disclosed herein, may contain: about 20% (w/w) or less, about 10% (w/w) or less, about 5% (w/w) or less, about 2% (w/w) or less, about 1% (w/w) or less, about 0.5% (w/w) or less, about 0.2% (w/w) or less, about 0.1% (w/w) or less, or about 0%, of any other solid state forms of the subject compound (Gefapixant, Gefapixant salt, or Gefapixant cocrystal, respectively), preferably as measured by XRPD. Thus, any of the disclosed crystalline forms of Gefapixant, Gefapixant salts, or Gefapixant cocrystals, described herein may be substantially free of any other solid state forms of the subject Gefapixant, Gefapixant salts, or Gefapixant cocrystals, respectively, and may contain greater than about 80% (w/w), greater than about 90% (w/w), greater than about 95% (w/w), greater than about 98% (w/w), greater than about 99% (w/w), or about 100% of the subject solid state form of the Gefapixant, Gefapixant salt, or Gefapixant cocrystal respectively.

The above crystalline polymorphs can be used to prepare other crystalline polymorphs of Gefapixant, Gefapixant salts, such as a citrate salt, Gefapixant co-crystals and their solid state forms.

The present disclosure encompasses a process for preparing other solid state forms of Gefapixant, Gefapixant salts, such as a citrate salt, Gefapixant co-crystals and their solid state forms thereof. The process includes preparing any one of the Gefapixant salts and solid state forms of Gefapixant by the processes of the present disclosure, and converting that salt to said other Gefapixant salt, for example Gefapixant citrate, or a crystalline form or a co-crystal thereof.

The present disclosure provides the above described crystalline polymorphs of Gefapixant or salts or co-crystals thereof for use in the preparation of pharmaceutical compositions comprising Gefapixant or salts or co-crystals thereof and/or crystalline polymorphs thereof.

The present disclosure also encompasses the use of crystalline polymorphs of Gefapixant or salts or co-crystals thereof of the present disclosure for the preparation of pharmaceutical compositions of crystalline polymorph Gefapixant or salts or co-crystals thereof and/or crystalline polymorphs thereof.

The present disclosure includes processes for preparing the above mentioned pharmaceutical compositions. The processes include combining any one or a combination of the crystalline polymorphs of Gefapixant or salts or co-crystals thereof of the present disclosure with at least one pharmaceutically acceptable excipient.

Pharmaceutical combinations or formulations of the present disclosure contain any one or a combination of the solid state forms of Gefapixant or salts or co-crystals thereof of the present disclosure. In addition to the active ingredient, the pharmaceutical formulations of the present disclosure can contain one or more excipients. Excipients are added to the formulation for a variety of purposes.

Diluents increase the bulk of a solid pharmaceutical composition, and can make a pharmaceutical dosage form containing the composition easier for the patient and caregiver to handle. Diluents for solid compositions include, for example, microcrystalline cellulose (e.g. Avicel®), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g. Eudragit®), potassium chloride, powdered cellulose, sodium chloride, sorbitol, and talc.

Solid pharmaceutical compositions that are compacted into a dosage form, such as a tablet, can include excipients whose functions include helping to bind the active ingredient and other excipients together after compression. Binders for solid pharmaceutical compositions include acacia, alginic acid, carbomer (e.g. carbopol), carboxymethylcellulose sodium, dextrin, ethyl cellulose, gelatin, guar gum, hydrogenated vegetable oil, hydroxyethyl cellulose, hydroxypropyl cellulose (e.g. Klucel®), hydroxypropyl methyl cellulose (e.g. Methocel®), liquid glucose, magnesium aluminum silicate, maltodextrin, methylcellulose, polymethacrylates, povidone (e.g. Kollidon®, Plasdone®), pregelatinized starch, sodium alginate, and starch.

The dissolution rate of a compacted solid pharmaceutical composition in the patient's stomach can be increased by the addition of a disintegrant to the composition. Disintegrants include alginic acid, carboxymethylcellulose calcium, carboxymethylcellulose sodium (e.g. Ac-Di-Sol®, Primellose®), colloidal silicon dioxide, croscarmellose sodium, crospovidone (e.g. Kollidon®, Polyplasdone®), guar gum, magnesium aluminum silicate, methyl cellulose, microcrystalline cellulose, polacrilin potassium, powdered cellulose, pregelatinized starch, sodium alginate, sodium starch glycolate (e.g. Explotab®), and starch.

Glidants can be added to improve the flowability of a non-compacted solid composition and to improve the accuracy of dosing. Excipients that can function as glidants include colloidal silicon dioxide, magnesium trisilicate, powdered cellulose, starch, talc, and tribasic calcium phosphate.

When a dosage form such as a tablet is made by the compaction of a powdered composition, the composition is subjected to pressure from a punch and dye. Some excipients and active ingredients have a tendency to adhere to the surfaces of the punch and dye, which can cause the product to have pitting and other surface irregularities. A lubricant can be added to the composition to reduce adhesion and ease the release of the product from the dye. Lubricants include magnesium stearate, calcium stearate, glyceryl monostearate, glyceryl palmitostearate, hydrogenated castor oil, hydrogenated vegetable oil, mineral oil, polyethylene glycol, sodium benzoate, sodium lauryl sulfate, sodium stearyl fumarate, stearic acid, talc, and zinc stearate.

Flavoring agents and flavor enhancers make the dosage form more palatable to the patient. Common flavoring agents and flavor enhancers for pharmaceutical products that can be included in the composition of the present disclosure include maltol, vanillin, ethyl vanillin, menthol, citric acid, fumaric acid, ethyl maltol, and tartaric acid.

Solid and liquid compositions can also be dyed using any pharmaceutically acceptable colorant to improve their appearance and/or facilitate patient identification of the product and unit dosage level.

In liquid pharmaceutical compositions of the present invention, Gefapixant or salts or co-crystals thereof and any other solid excipients can be dissolved or suspended in a liquid carrier such as water, vegetable oil, alcohol, polyethylene glycol, propylene glycol, or glycerin.

Liquid pharmaceutical compositions can contain emulsifying agents to disperse uniformly throughout the composition an active ingredient or other excipient that is not soluble in the liquid carrier. Emulsifying agents that can be useful in liquid compositions of the present invention include, for example, gelatin, egg yolk, casein, cholesterol, acacia, tragacanth, chondrus, pectin, methyl cellulose, carbomer, cetostearyl alcohol, and cetyl alcohol.

Liquid pharmaceutical compositions of the present invention can also contain a viscosity enhancing agent to improve the mouth-feel of the product and/or coat the lining of the gastrointestinal tract. Such agents include acacia, alginic acid bentonite, carbomer, carboxymethylcellulose calcium or sodium, cetostearyl alcohol, methyl cellulose, ethylcellulose, gelatin guar gum, hydroxyethyl cellulose, hydroxypropyl cellulose, hydroxypropyl methyl cellulose, maltodextrin, polyvinyl alcohol, povidone, propylene carbonate, propylene glycol alginate, sodium alginate, sodium starch glycolate, starch tragacanth, xanthan gum and combinations thereof.

Sweetening agents such as sorbitol, saccharin, sodium saccharin, sucrose, aspartame, fructose, mannitol, and invert sugar can be added to improve the taste.

Preservatives and chelating agents such as alcohol, sodium benzoate, butylated hydroxyl toluene, butylated hydroxyanisole, and ethylenediamine tetraacetic acid can be added at levels safe for ingestion to improve storage stability.

According to the present disclosure, a liquid composition can also contain a buffer such as gluconic acid, lactic acid, citric acid, or acetic acid, sodium gluconate, sodium lactate, sodium citrate, or sodium acetate. Selection of excipients and the amounts used can be readily determined by the formulation scientist based upon experience and consideration of standard procedures and reference works in the field.

The solid compositions of the present disclosure include powders, granulates, aggregates, and compacted compositions. The dosages include dosages suitable for oral, buccal, rectal, parenteral (including subcutaneous, intramuscular, and intravenous), inhalant, and ophthalmic administration. Although the most suitable administration in any given case will depend on the nature and severity of the condition being treated, in embodiments the route of administration is oral. The dosages can be conveniently presented in unit dosage form and prepared by any of the methods well-known in the pharmaceutical arts.

Dosage forms include solid dosage forms like tablets, powders, capsules, suppositories, sachets, troches, and lozenges, as well as liquid syrups, suspensions, and elixirs.

The dosage form of the present disclosure can be a capsule containing the composition, such as a powdered or granulated solid composition of the disclosure, within either a hard or soft shell. The shell can be made from gelatin and optionally contain a plasticizer such as glycerin and/or sorbitol, an opacifying agent and/or colorant.

The active ingredient and excipients can be formulated into compositions and dosage forms according to methods known in the art.

A composition for tableting or capsule filling can be prepared by wet granulation. In wet granulation, some or all of the active ingredients and excipients in powder form are blended and then further mixed in the presence of a liquid, typically water, that causes the powders to clump into granules. The granulate is screened and/or milled, dried, and then screened and/or milled to the desired particle size. The granulate can then be tableted, or other excipients can be added prior to tableting, such as a glidant and/or a lubricant.

A tableting composition can be prepared conventionally by dry blending. For example, the blended composition of the actives and excipients can be compacted into a slug or a sheet and then comminuted into compacted granules. The compacted granules can subsequently be compressed into a tablet.

As an alternative to dry granulation, a blended composition can be compressed directly into a compacted dosage form using direct compression techniques. Direct compression produces a more uniform tablet without granules. Excipients that are particularly well suited for direct compression tableting include microcrystalline cellulose, spray dried lactose, dicalcium phosphate dihydrate, and colloidal silica. The proper use of these and other excipients in direct compression tableting is known to those in the art with experience and skill in particular formulation challenges of direct compression tableting.

A capsule filling of the present disclosure can include any of the aforementioned blends and granulates that were described with reference to tableting, but they are not subjected to a final tableting step.

A pharmaceutical formulation of Gefapixant or salts or co-crystals thereof can be administered. Gefapixant or salts or co-crystals thereof may be formulated for administration to a mammal, in embodiments to a human, by injection. Gefapixant or salts or co-crystals thereof can be formulated, for example, as a viscous liquid solution or suspension, such as a clear solution, for injection. The formulation can contain one or more solvents. A suitable solvent can be selected by considering the solvent's physical and chemical stability at various pH levels, viscosity (which would allow for syringeability), fluidity, boiling point, miscibility, and purity. Suitable solvents include alcohol USP, benzyl alcohol NF, benzyl benzoate USP, and Castor oil USP. Additional substances can be added to the formulation such as buffers, solubilizers, and antioxidants, among others. Ansel et al., Pharmaceutical Dosage Forms and Drug Delivery Systems, 7th ed.

The crystalline polymorphs of Gefapixant or salts or co-crystals thereof and the pharmaceutical compositions and/or formulations of Gefapixant or salts or co-crystals thereof of the present disclosure can be used as medicaments, in embodiments in the treatment of chronic cough, asthma, interstitial cystitis, musculoskeletal pain, pelvic pain or sleep apnea syndrome, and particularly chronic cough.

The present disclosure also provides methods of treating chronic cough, asthma, interstitial cystitis, musculoskeletal pain, pelvic pain or sleep apnea syndrome, and particularly chronic cough by administering a therapeutically effective amount of any one or a combination of the crystalline polymorphs of Gefapixant or salts or co-crystals thereof of the present disclosure, or at least one of the above pharmaceutical compositions and/or formulations, to a subject in need of the treatment.

Having thus described the disclosure with reference to particular preferred embodiments and illustrative examples, those in the art can appreciate modifications to the disclosure as described and illustrated that do not depart from the spirit and scope of the disclosure as disclosed in the specification. The Examples are set forth to aid in understanding the disclosure but are not intended to, and should not be construed to limit its scope in any way.

### Powder X-Ray Diffraction (“XRPD”) Method

Sample after being powdered in a mortar and pestle is applied directly on a silicon plate holder. The X-ray powder diffraction pattern was measured with Philips X'Pert PRO X-ray powder diffractometer, equipped with Cu irradiation source=1.54184 Å (Ångström), X'Celerator (2.022° 2θ) detector. Scanning parameters: angle range: 3-40 deg., step size 0.0167, time per step 37 s, continuous scan. The described peak positions were determined without using silicon powder as an internal standard.

### Differential Scanning Calorimetry (“DSC”) Method

DSC measurements were done using TA Instruments Discovery, DSC unit. 1-3 mg of sample was weighted in pan, hermetically closed with the pin hole. Sample was purged with 50 ml/min of nitrogen flow and heated in the range of 25-350° C., with heating rate of 10° C./min.

### Thermal Gravimetric Analysis (“TGA”) Method

TGA measurements were done using TA Instruments Discovery, TG unit. 5-10 mg of sample was weighted in open aluminum pan. Sample was purged with 50 ml/min of nitrogen flow and heated in the range of 25-350° C., with heating rate of 10° C./min.

### EXAMPLES

### Preparation of Starting Materials

Gefapixant can be prepared according to methods known from the literature, for example International Publication No. WO 2005/095359. Gefapixant form A and Gefapixant Citrate form A can be prepared according to methods known from the literature, for example International Publication No. WO 2018/118668.

For example, Gefapixant form A can be prepared according to the following procedure:

Gefapixant (50 mg) was dissolved in a solvent mixture ethanol:water (total volume 4 ml, ratio 1:1) at a temperature of about 77° C. The obtained solution was allowed to room temperature and crystallization occurred. The obtained suspension was vacuum filtered and a sample was analyzed by XRPD. Gefapixant form A was obtained.

The products in the following examples may be dried by air (e.g. on the filter paper), e.g. at a temperature of about 20° C. to about 25° C. Optionally, Forms M1, Ic, It, Im, If, and L1 may be dried under vacuum, for example at a temperature of about 25° C. to about 80° C., about 30° C. to about 70° C., about 40° C. to about 60° C., until the drying is completed, e.g. to a constant weight.

### Example 1: Preparation of Gefapixant Form 1

Gefapixant form 2 (3.5 mg) was placed in pin hole aluminum pan. Sample was subjected to thermal treatment in DSC Discovery TA instruments by heating of the sample by heating rate 10° C./minute up to temperature of 99° C., Isothermal heating for 15 minutes at 99° C., and cooled to room temperature. The obtained solid was analyzed by XRPD. Form 1 was obtained. An XRPD pattern is shown in FIG. 1.

### Example 2: Preparation of Gefapixant Form 2

500 mg of Gefapixant was dissolved in 26 ml of solvent mixture THF:Water (1:1) at temperature of about 60° C. The solution was cooled down slowly and crystallization was occurred at temperature of about 10° C. The suspension was stirred for period of about 1 hour and filtered under vacuum at temperature of 20° C. to about 25° C. The obtained solid was washed with 6 ml of clear solvent mixture THF:Water (1:1) and was analyzed by XRPD. Form 2 was obtained. An XRPD pattern is shown in FIG. 2.

### Example 3: Preparation of Gefapixant Form 4

1800 mg of Gefapixant base was dissolved in 35 ml of acetic acid at temperature of about 85° C. The solution was cooled and crystallization was occurred at temperature of about 60° C. The suspension was cooled to temperature of about 20° C. to about 25° C. and filtered under vacuum at temperature of about 20° C. to about 25° C. The obtained solid was analyzed by XRPD. Form 4 was obtained. An XRPD pattern is shown in FIG. 3.

### Example 4a: Preparation of Gefapixant Citrate Form T1

47.9 mg of Gefapixant and 52.1 mg of citric acid (2.2 eq.) were suspended in 1 ml of THF at temperature of about of about 20° C. to about 25° C. After 24 hours, the suspension was filtered under vacuum at filtered under vacuum at temperature of about 20° C. to about 25° C. The obtained solid was analyzed by XRPD. Form T1 was obtained. An XRPD pattern is shown in FIG. 4.

### Example 4b: Preparation of Gefapixant Citrate Form T1

47.9 mg of Gefapixant (form A) and 52.1 mg of citric acid (2.2 eq.) were suspended in 1 ml of THF at temperature of about of about 20° C. to about 25° C. After 24 hours, the suspension was filtered under vacuum at temperature of about 20° C. to about 25° C. The obtained solid was analyzed by XRPD confirming that Form T1 as per FIG. 4 was obtained.

### Example 5: Preparation of Gefapixant Citrate Form E1

57.1 mg of Gefapixant Form 4 and 42.9 mg of citric acid (2.2 eq.) were suspended in 1 ml of Ethanol at temperature of about 20° C. to about 25° C. After 15 days, the suspension was filtered under vacuum at temperature of about 20° C. to about 25° C. The obtained solid was analyzed by XRPD. Form E1 was obtained. An XRPD pattern is shown in FIG. 5.

### Example 6. Preparation of Gefapixant Maleate Form M1

70 mg of Gefapixant form A and 30 mg of maleic acid (2 eq.) were suspended in 1 ml of ethanol at temperature of about 20° C. to about 25° C. After 24 hours, the suspension was filtered under vacuum at temperature of about 20° C. to about 25° C. The obtained solid was analyzed by XRPD. Form M1 was obtained. An XRPD pattern is shown in FIG. 6.

### Example 7: Preparation of Amorphous Gefapixant Citrate

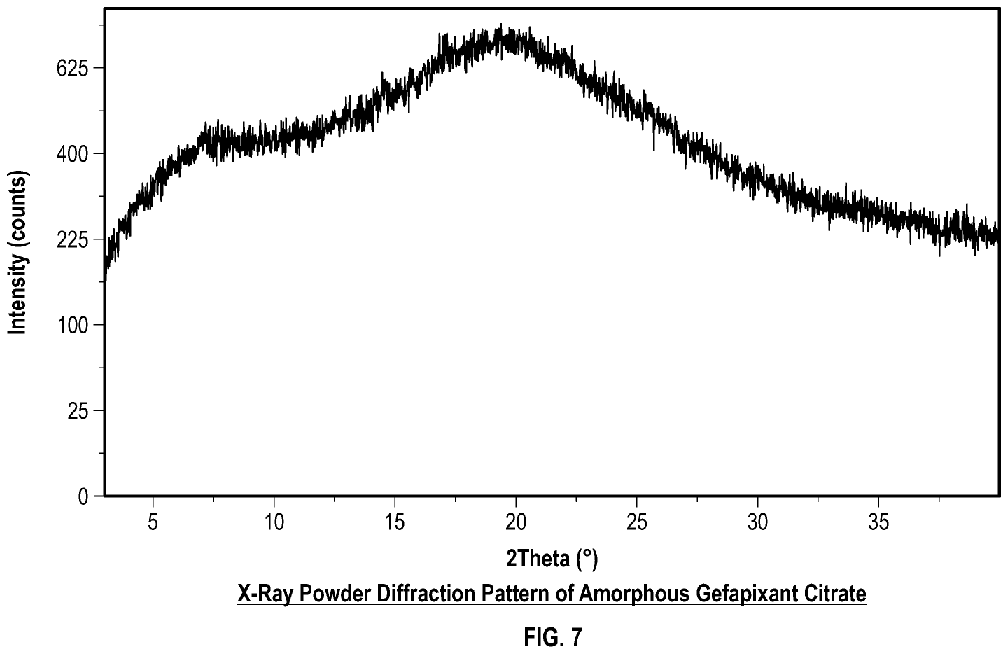

5.912 g of Gefapixant citrate Form E1 was dried at temperature of about 100° C. for period of about 2 hours in vacuum dryer. Sample was analyzed by XRPD. Gefapixant citrate amorphous was obtained An XRPD pattern is shown in FIG. 7.

### Example 8: Preparation of Gefapixant Camsylate Form Ic

54 mg of Gefapixant form A and 46 mg of camphor-10-sulfonic acid (2 eq.) were suspended in 1 ml of ethanol at about 20° C. to about 25° C. Suspension was vacuum filtered after 24 hours at temperature of about 20° C. to about 25° C. The obtained solid was analyzed by XRPD. Gefapixant camsylate Form Ic was obtained. An XRPD pattern is shown in FIG. 8.

### Example 9: Preparation of Gefapixant Camsylate Form Ic

500 mg of Gefapixant form A was suspended in 6 ml of 2-propanol at temperature of about 60° C. Water solution of camphor-10-sulfonic acid (3.5 ml, 2 eq.) was added drop wise in suspension at temperature of about 60° C. Suspension was dissolved at temperature of about 60° C. and slow cooled to about 20° C. to about 25° C. Crystallization was occurred after 2 hours of stirring at temperature of about 20° C. to about 25° C. Suspension was vacuum filtered after 2 hours at temperature of about 20° C. to about 25° C. The obtained solid was analyzed by XRPD. Gefapixant camsylate Form Ic was obtained.

### Example 10: Preparation of Gefapixant Tosylate Form it

500 mg of Gefapixant form A was suspended in 6.7 ml of 2-propanol at temperature of about 50° C. Water solution of p-toluenesulfonic acid monohydrate (3.6 ml, 2 eq.) was added drop wise in suspension at temperature of about 50° C. Suspension was dissolved at temperature of about 50° C. and stirred for period of about 1 hour. The solution was slow cooled to temperature of about 0° C. Crystallization was occurred after 2 hours of stirring at temperature of about 10° C. Suspension was vacuum filtered after 2 hours at temperature of about 20° C. to about 25° C. The obtained solid was analyzed by XRPD. Gefapixant tosylate Form It was obtained. An XRPD pattern is shown in FIG. 9.

### Example 11: Preparation of Gefapixant Hemiedisylate Form Ie

500 mg of Gefapixant form A was suspended in 6.7 ml of 2-propanol at temperature of about 50° C. Water solution of 1,2-ethanedisulfonic acid dihydrate (4.3 ml, 2 eq.) was added drop wise in suspension at temperature of about 50° C. Suspension was dissolved at a temperature of about 50° C. and slow cooled to temperature of about 20° C. to about 25° C. Crystallization was occurred after 2 hours of stirring at temperature of about 20° C. to about 25° C. Suspension was vacuum filtered after 2 hours at temperature of about 20° C. to about 25° C. The obtained solid was analyzed by XRPD. Gefapixant hemiedisylate Form Ie was obtained. An XRPD pattern is shown in FIG. 10.

### Example 12: Preparation of Gefapixant Edisylate Form IIe

500 mg of Gefapixant hemiedisylate form Ie was suspended in 6 ml of 2-Propanol at temperature of about 50° C. Water solution of 1,2-ethanedisulfonic acid dihydrate (3 ml, 0.5 eq) was added drop wise in suspension at temperature of about 50° C. Suspension was dissolved at temperature of about 50° C. and slow cooled to temperature of about 20° C. to about 25° C. Crystallization was occurred after 1 hour of stirring at temperature of about 20° C. to about 25° C. Suspension was vacuum filtered after 2 hours at temperature of about 20° C. to about 25° C. The obtained solid was analyzed by XRPD. Gefapixant hemiedisylate Form IIe was obtained. An XRPD pattern is shown in FIG. 11.

### Example 13: Preparation of Gefapixant Malonate Form Im

500 mg of Gefapixant form A was suspended in 10 ml of 2-propanol at temperature of about 50° C. Water solution of malonic acid (7 ml, 3 eq) was added drop wise in suspension at temperature of about 50° C. Suspension was dissolved at temperature of about 50° C. and slow cooled to temperature of about 20° C. to about 25° C. Crystallization had not occurred after 3 hours of stirring, then solution was evaporated on rotavapour (50° C., 0 mbar) and oil was obtained. 10 ml of water was added in oil and stirred for period of about 6 hours. After 6 hours, crystallization occurred. Suspension was vacuum filtered at temperature of about 20° C. to about 25° C. The obtained solid was analyzed by XRPD. Gefapixant malonate Form Im was obtained. An XRPD pattern is shown in FIG. 12.

### Example 14: Preparation of Gefapixant Fumarate Form if

250 mg of Gefapixant form A and 164.37 mg (2 eq.) of fumaric acid were dissolved in 5.5 ml of solvent mixture Ethanol:Water (ratio 2:1) at temperature of about 61° C. The solution was stirred for a period of about 20 minutes at temperature of about 61° C. and slow cooled to temperature of about 20° C. to about 25° C. Crystallization occurred at a temperature of about 43° C. Suspension was cooled to temperature of about 20° C. to about 25° C. and stirred for period of about 2 hours. Suspension was vacuum filtered at temperature of about 20° C. to about 25° C. The obtained solid was analyzed by XRPD. Gefapixant fumarate form If was obtained. An XRPD pattern is shown in FIG. 13.

### Example 15: Preparation of Crystalline Gefapixant Citrate:L-Lysine Complex, Form L1

275 mg of Gefapixant citrate form A and 225 mg of L-Lysine (3 eq.) were suspended in 5 ml of Methyl isobutyl ketone (MIBK) at temperature of about 40° C. After 24 hours of stirring, suspension was vacuum filtered at temperature of about 20° C. to about 25° C. The obtained solid was analyzed by XRPD. Crystalline Gefapixant citrate:L-Lysine complex, Form L1 was obtained. An XRPD pattern is shown in FIG. 14.

### Example 16: Preparation of Gefapixant Tosylate Form IIt

30 mg of Gefapixant tosylate form It was put in small vial and exposed to solvent vapour (2 ml of 2-propanol) in big glass vial at room temperature. The obtained solid was analyzed by XRPD after 7 days. Gefapixant tosylate form IIt was obtained. An XRPD pattern is shown in FIG. 15.

### Example 17: Preparation of Gefapixant Fumarate Form IIf

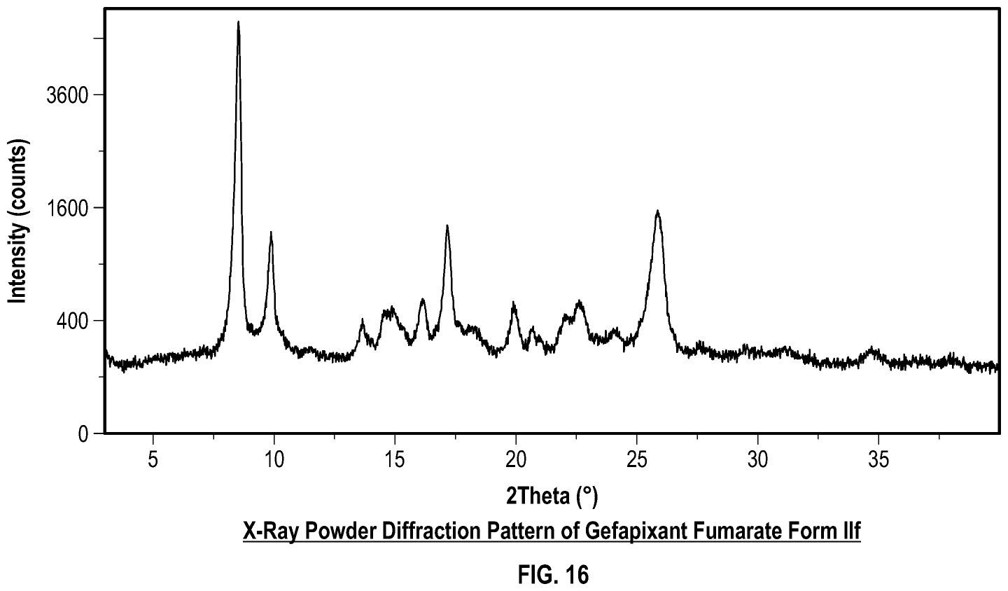

Gefapixant fumarate form If (500 mg) was vacuum dried at temperature of about 100° C. for period of about 3 hours in vacuum dryer. The obtained solid was analyzed by XRPD. Gefapixant fumarate form IIf was obtained. An XRPD pattern is shown in FIG. 16.

1. Crystalline Form M1 of Gefapixant maleate characterized by data selected from one or more of the following:
a) an X-ray powder diffraction pattern substantially as depicted in FIG. 6; or
b) an X-ray powder diffraction pattern having peaks at 10.0, 13.2, 15.5, 18.4 and 22.3 degrees 2-theta±0.2 degrees 2-theta.

2. Crystalline Form M1 of Gefapixant maleate according to claim 1, characterized by an X-ray powder diffraction pattern having peaks at 10.0, 13.2, 15.5, 18.4 and 22.3 degrees 2-theta±0.2 degrees 2-theta, and also having any one, two, three, four or five additional peaks selected from 10.5, 16.6, 20.1, 21.4 and 24.0 degrees 2-theta±0.2 degrees 2-theta.

3. Crystalline Form M1 of Gefapixant maleate according to claim 1, characterized by an X-ray powder diffraction pattern having peaks at 10.0, 10.5, 13.2, 15.5, 16.6, 18.4, 20.1, 21.4, 22.3 and 24.0 degrees 2-theta±0.2 degrees 2-theta.

4. Crystalline Form M1 of Gefapixant maleate according to claim 1, characterized by an X-ray powder diffraction pattern having peaks according to Table 1:at 10.0, 10.5, 11.8, 11.9, 13.2, 13.9, 15.5, 16.4, 16.6, 18.4, 19.3, 19.5, 20.1, 20.6, 20.9, 21.4, 21.9, 22.3, 22.5, 24.0, 24.3, 24.6, 25.0, 25.5, 25.7, 26.0, 26.3, 26.4, 27.0, 27.8, 30.3, 30.7, 31.1, 33.2, and 33.4 degrees 2-theta±0.2 degrees 2-theta.

5. Crystalline Form M1 of Gefapixant maleate according to claim 1, which is further characterized by having a DSC melting onset of: about 226.0° C. to about 232.0° C., or 226.0° C. to about 231.0° C., or from about 227.0° C. to about 232.0° C., from about 227.0° C. to about 231.0° C., about 229.0° C.±1.0° C., or about 229.0° C., or a DSC thermogram substantially as depicted in FIG. 17.

6. Crystalline Form M1 of Gefapixant maleate according to claim 1, which is further characterized by TGA thermogram substantially as depicted in FIG. 18.

7. Crystalline Form M1 of Gefapixant maleate according to claim 1, which is an anhydrous form.

8. Crystalline Form M1 of Gefapixant maleate according to claim 1, which contains no more than about 20%, no more than about 10%, no more than about 5%, no more than about 20% of any other crystalline forms of Gefapixant maleate; and/or no more than about 20% of amorphous Gefapixant maleate.

9. Crystalline Form Ic of Gefapixant camsylate characterized by data selected from one or more of the following:
a) an X-ray powder diffraction pattern substantially as depicted in FIG. 8; or
b) an X-ray powder diffraction pattern having peaks at 6.0, 18.0, 22.6 and 24.4 degrees 2-theta±0.2 degrees 2-theta.

10. Crystalline Form Ic of Gefapixant camsylate according to claim 9, characterized by an X-ray powder diffraction pattern having peaks at 6.0, 18.0, 22.6 and 24.4 degrees 2-theta±0.2 degrees 2-theta, and also having any one, two, three, four or five additional peaks selected from 10.9, 21.8, 24.9 and 22.1 degrees 2-theta±0.2 degrees 2-theta.

11. Crystalline Form Ic of Gefapixant camsylate according to claim 9, characterized by an X-ray powder diffraction pattern having peaks at 6.0, 10.9, 18.0, 21.8, 22.1, 22.6, 24.4 and 24.9 degrees 2-theta±0.2 degrees 2-theta.

12-16. (canceled)

17. Crystalline Form Ie of Gefapixant hemiedisylate characterized by data selected from one or more of the following:
a) an X-ray powder diffraction pattern substantially as depicted in FIG. 10; or
b) an X-ray powder diffraction pattern having peaks at 14.4, 15.9, 19.9, 21.6, and 27.6 degrees 2-theta±0.2 degrees 2-theta.

18. Crystalline Form Ie of Gefapixant hemiedisylate according to claim 17, characterized by an X-ray powder diffraction pattern having peaks at 14.4, 15.9, 19.9, 21.6, and 27.6 degrees 2-theta±0.2 degrees 2-theta, and also having any one, two, three, four or five additional peaks selected from 11.4, 19.2, 21.1, 22.7 and 28.1 degrees 2-theta±0.2 degrees 2-theta.

19. Crystalline Form Ie of Gefapixant hemiedisylate according to claim 17, characterized by an X-ray powder diffraction pattern having peaks at 11.4, 14.4, 15.9, 19.2, 19.9, 21.1, 21.6, 22.7, 27.6 and 28.1 degrees 2-theta±0.2 degrees 2-theta.

20-23. (canceled)

24. A pharmaceutical composition comprising a crystalline form according to claim 1.

25. A pharmaceutical formulation comprising a crystalline form according to claim 1 and at least one pharmaceutically acceptable excipient.

26. A process for preparing a pharmaceutical formulation comprising combining a crystalline form according to claim 1 with at least one pharmaceutically acceptable excipient.

27. (canceled)

28. A medicament comprising the crystalline form according to claim 1.

29. (canceled)

30. A method of treating chronic cough, asthma, interstitial cystitis, musculoskeletal pain, pelvic pain or sleep apnea syndrome, and particularly chronic cough, comprising administering a therapeutically effective amount of a crystalline form according to claim 1 to a subject in need of the treatment.

31. (canceled)

32. (canceled)

33. A process for preparing solid state form of Gefapixant, Gefapixant co-crystals and solid state forms thereof, or Gefapixant salts, co-crystal or solid state form thereof, comprising preparing a crystalline form according to claim 1, and converting it to another solid state form of Gefapixant, or to Gefapixant co-crystal and solid state forms thereof, or to Gefapixant salt and co-crystal or solid state form thereof.
